SEQUENCE LISTING (59 sequences)

SEQ ID NO: 1 - protein, 21 aa, synthetic construct
DKINHESNEKAAADSDNQKAE

SEQ ID NO: 2 - protein, 21 aa, synthetic construct
DKINHESNSKAAATSDNQKAL

SEQ ID NO: 3 - protein, 21 aa, synthetic construct
DKGASGTLNKSGIVVGITKAQ

SEQ ID NO: 4 - protein, 21 aa, synthetic construct
NKGASGTLNKSGIVVGITKAQ

SEQ ID NO: 5 - protein, 21 aa, synthetic construct
DNIDKEHNDEKAADSDKEHDE

SEQ ID NO: 6 - protein, 21 aa, synthetic construct
NSITKEHNDEKAADSDKQHAG

SEQ ID NO: 7 - protein, 21 aa, synthetic construct
NSGAKGTLNQKGIVVGKSVAQ

SEQ ID NO: 8 - protein, 21 aa, synthetic construct
NSGAKGTLNQKGIVHGKSVAQ

SEQ ID NO: 9-13 - skipped (5 entries)

SEQ ID NO: 14 - protein, 7 aa, synthetic construct
PKKKRKV

SEQ ID NO: 15 - protein, 16 aa, synthetic construct
KRPAATKKAGQAKKKK

SEQ ID NO: 16 - protein, 17 aa, synthetic construct
KRPMNAFIVWSRDQRRK

SEQ ID NO: 17 - protein, 5 aa, synthetic construct
RPRRK

SEQ ID NO: 18 - protein, 20 aa, synthetic construct
SSNFGPMKGGNRFFRSSGPY

SEQ ID NO: 19 - protein, 27 aa, synthetic construct
RSGGNHRRNGRGGRGGYNRRNNGYHPY

SEQ ID NO: 20 - protein, 16 aa, synthetic construct
LRLTLLELVRRLNGNG

SEQ ID NO: 21 - protein, 10 aa, synthetic construct
GKISKHWTGI

SEQ ID NO: 22 - protein, 7 aa, synthetic construct
SKKSLED

SEQ ID NO: 23 - protein, 12 aa, synthetic construct
RQARRNRRRRWR

SEQ ID NO: 24 - protein, 12 aa, synthetic construct
GRKKRRQRRRAP

SEQ ID NO: 25 - protein, 18 aa, synthetic construct
MPKTRRRPRRSQRKRPPT

SEQ ID NO: 26-28 - skipped (3 entries)

SEQ ID NO: 29 - protein, 12 aa, synthetic construct
GYGPKKKRKVGG

SEQ ID NO: 30 - protein, 5 aa, synthetic construct
GGGGS

SEQ ID NO: 31 - protein, 10 aa, synthetic construct
GGGGSGGGGS

SEQ ID NO: 32 - protein, 15 aa, synthetic construct
GGGGSGGGGSGGGGS

SEQ ID NO: 33 - protein, 20 aa, synthetic construct
GGGGSGGGGSGGGGSGGGGS

SEQ ID NO: 34 - protein, 25 aa, synthetic construct
GGGGSGGGGSGGGGSGGGGSGGGGS

SEQ ID NO: 35 - protein, 30 aa, synthetic construct
GGGGSGGGGSGGGGSGGGGSGGGGSGGGGS

SEQ ID NO: 36 - protein, 35 aa, synthetic construct
GGGGSGGGGSGGGGSGGGGSGGGGSGGGGSGGGGS

SEQ ID NO: 37 - protein, 25 aa, synthetic construct
GKPGSGKPGSGKPGSGKPGSGKPGS

SEQ ID NO: 38 - protein, 30 aa, synthetic construct
GKPGSGKPGSGKPGSGKPGSGKPGSGKPGS

SEQ ID NO: 39 - protein, 21 aa, synthetic construct
DRIRKERNDEKAADSRKRRRE

SEQ ID NO: 40 - protein, 30 aa, synthetic construct
DNKDIEHKDENAKDSDAKHNDEKAADSKSA

SEQ ID NO: 41 - protein, 32 aa, synthetic construct
DNIKIEHNKENADKSDAHKNDEAKADSSKEHG

SEQ ID NO: 42 - protein, 35 aa, synthetic construct
DNIDKEHNDEKAADSDKEHNDEKAADSKSEHDKAG

SEQ ID NO: 43 - protein, 41 aa, synthetic construct
DNIKDEDNKEAADKSADHKNDEAKASSAKEHADKNDEAKAG

SEQ ID NO: 44 - protein, 46 aa, synthetic construct
DNKDAEAKDEAAKDSADKHNDEKAAASKSEHAKANDEKAADSKVAG

SEQ ID NO: 45 - protein, 46 aa, synthetic construct
DNKDSENKDEAAKDSDDKHNDEAKASSAKEHADKNDEAKAGSAKAG

SEQ ID NO: 46 - protein, 122 aa, synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSS

SEQ ID NO: 47 - protein, 8 aa, synthetic construct
GITFDDAV

SEQ ID NO: 48 - protein, 8 aa, synthetic construct
ISSNSGYI

SEQ ID NO: 49 - protein, 15 aa, synthetic construct
VKGLYSNPRGGAFDI

SEQ ID NO: 50 - protein, 110 aa, synthetic construct
SYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 51 - protein, 6 aa, synthetic construct
NIGTKS

SEQ ID NO: 52 - skipped

SEQ ID NO: 53 - protein, 12 aa, synthetic construct
QVWDSRSDHLWV

SEQ ID NO: 54 - protein, 253 aa, synthetic construct
EVQLVESGGGLIQPGRSLRLSCAASGITFDDAVMHWVRQAPGKGLEWVAGISSNSGYIGYADSVKGRFTISRDNAKNSLYLQMNRLRAEDTAVYYCVKGLYSNPRGGAFDIWGQGTMVTVSSASTGGGGSGGGGSGGGGSVHSSYVLTQPPSVSVAPGQTATITCGGNNIGTKSVHWYQQKPGQAPVLVVYADSDRPSGIPERVSGSNSGNTATLTISRVEAGDEADYYCQVWDSRSDHLWVFGGGTKLTVLG

SEQ ID NO: 55 - protein, 8 aa, synthetic construct
PPRSKKRI

SEQ ID NO: 56 - protein, 28 aa, synthetic construct
DKINHESNEKAAADSDNQKAEGGGNPGY

SEQ ID NO: 57 - protein, 28 aa, synthetic construct
DNIDKEHNDEKAADSDKEHDEGGGNPGY

SEQ ID NO: 58 - protein, 28 aa, synthetic construct
NSITKEHNDEKAADSDKQHAGGGGNPGY

SEQ ID NO: 59 - protein, 28 aa, synthetic construct
NSGAKGTLNQKGIVHGKSVAQGGGNPGY

(19) United States
(12) Patent Application Publication
(10) Pub. No.: US 2026/0109780 A1
(43) Pub. Date: Apr. 23, 2026

(54) DOCKING PEPTIDES AND RELATED ANTIBODY-BASED DRUG CONJUGATES

(71) Applicant: Sonnet BioTherapeutics, Inc., Princeton, NJ (US)

(72) Inventors: John K. Cini, Blairstown, NJ (US); Nick Evans, Hertford (GB)

(21) Appl. No.: 19/186,382

(22) Filed: Apr. 22, 2025

Related U.S. Application Data
(60) Provisional application No. 63/709,765, filed on Oct. 21, 2024.

(51) Int. Cl. C07K 16/32; A61K 47/66; A61K 47/68; A61P 35/00

(52) U.S. Cl. CPC C07K 16/32; A61K 47/66; A61K 47/68031; A61K 47/6855; A61K 47/6889; A61P 35/00; C07K 2317/622; C07K 2319/09

(57) ABSTRACT

Provided herein are novel docking peptides and antibody-based drug conjugates that include such docking peptides. In embodiments, the subject docking peptide allows for the controlled attachment of one or more drug moieties and a tumor targeting moiety that allows for the targeting of the drug moiety to the tumor microenvironment.

In some embodiments, the antibody-based drug conjugates provided herein advantageously allows for enhanced targeted delivery of a drug payload having a controlled drug-to-antibody ratio (DAR) to a target site.

### PRIORITY

This application claims priority to U.S. Patent Application No. 63/709,765, filed Oct. 21, 2024, which is expressly incorporated by reference in its entirety.

### SEQUENCE LISTING

The instant application contains a Sequence Listing which has been submitted electronically in XML file format and is hereby incorporated by reference in its entirety. Said XML copy, created on Jan. 2, 2025, is named “116076-5023-WO-Sequence Listing” and 49,178 bytes in size.

### BACKGROUND

In classic chemotherapy, the mainstay of anticancer treatment, cytotoxic drugs are delivered orally or systemically, but the efficacy of such treatment is often compromised by extensive side effects and limited drug exposure resulting from systemic delivery. Moreover, the limited selectivity against cancer cells in chemotherapy treatments leads to a small therapeutic window, thus limiting efficacy. Monoclonal antibodies have proven to play an important role in cancer treatment, with therapeutics such as trastuzumab, bevacizumab, pertuzumab, cetuximab, panitumumab, gemtuzumab, alemtuzumab and rituximab becoming the standard of care in selected solid tumors, leukemias and lymphomas.

Antibody-drug conjugates (ADCs) promise to provide highly selective and highly cytotoxic cancer treatment with an increased therapeutic window. ADCs utilize the selectivity of antibodies or antibody fragments to deliver potent anti-cancer agents that are often too toxic to be dosed systematically alone. Early ADCs encountered major obstacles including, low blood residency time, low penetration capacity to the tumor microenvironment, low payload potency, immunogenicity, unusual off-target toxicity, drug resistance, limiting control of the number of drugs per antibody ratio (DAR) and the lack of stable chemical linkage in blood circulation. Although extensive research has been conducted to overcome these issues, the ADC-based therapies are still far from having high-efficient clinical outcomes. Thus, there remains a need for novel cancer treatments that improve upon the above-referenced obstacles, particularly pertaining to antibody-based drug conjugates.

### SUMMARY

Provided herein are novel docking peptides and antibody-based drug conjugates that include such docking peptides. In embodiments, the docking peptide advantageously allows for the efficient and controlled attachment of one or more drug moieties. In some embodiments, the subject antibody-based drug conjugates are made in a two-step process by first conjugating payloads to the subject docking peptide (see FIG. 4, step1). The docking peptide linked with the payloads is then attached to an antibody-based targeting moiety (FIG. 4, step 2). This ADC complex allows for the targeting of the drug moiety to the tumor microenvironment. In some embodiments, the antibody-based drug conjugates provided herein advantageously allows for enhanced targeted delivery of a drug payload having a controlled drug-to-antibody ratio (DAR) to a tumor site. Aspects of the antibody-based drug conjugates are discussed in further detail herein.

In one aspect provided herein, is an antibody-based drug conjugate. The antibody-based drug conjugate comprises: a) a targeting moiety; and b) a drug conjugate moiety. The drug moiety comprises: i) a docking peptide; and ii) one or more drug moieties attached to the docking peptide. The targeting moiety is attached to the drug conjugate moiety by a linker. In some embodiments, the docking peptide comprises the amino acid sequence of any one of SEQ ID NOs: 1-8 and 39-45. In some embodiments, the docking peptide comprises the amino acid sequence of SEQ ID NO:5. In some embodiments, the targeting moiety is a tumor targeting moiety. In some embodiments, the tumor targeting moiety binds to one or more of the following tumor antigens: CD33, CD30, HER2, HER3, CD22, CD79b, NaPi2b, glycoprotein NMB, CD19, CD138, PSMA, CEA, guanlyl cyclase C, CD198, EGFR, CD52, CD74, FOLR1, CD37, mesothelin, CECAMS, LAMP1, GPNMB, CD56, TROP2, Mucin 1, STEAP1, Mesotehlin, Nectin 4, ENP3, guanylyl cyclase C (GCC), SLC44A4, NaPi2b, CD70 (TNFSF7), Cap, 5T4, SLTRK6, SC-16, LIV-1 (ZIP6), and P-Cadherin.

In some embodiments, the tumor targeting moiety comprises one or more anti-tumor antigen scFvs. In exemplary embodiments, the targeting moiety comprises two anti-tumor antigen scFvs arranged in tandem. In some embodiments, the anti-tumor scFvs each bind to a different tumor antigen.

In some embodiments, the antibody-based drug conjugate further comprises an anti-human serum albumin scFv. In some embodiments, the anti-human serum albumin scFv is attached to the targeting moiety and the drug conjugate moieties by linkers. In some embodiments, the anti-human serum albumin scFv has the amino acid sequence of SEQ ID NO: 38.

In some embodiments, the tumor targeting moiety comprises a first scFv, and a second scFv, Vhh or Fab that each bind to a tumor target antigen, and a third scFv that binds to human serum albumin.

In some embodiments, each of the one or more drug moieties comprises a nuclear localization signal.

In some embodiments, the drug conjugate moiety further comprises an endosomal-lysosomal sorting signal.

In exemplary embodiments, the one or more drug moieties are each attached to a lysine residue of the docking peptide by a linker. In some embodiments, the linker is a cleavable linker. In some embodiments, the cleavable linker is a cathepsin B cleavable linker. In some embodiments, the linker is selected from a hydrazone linker, a peptide linker, a disulfide linker and a thioether linker.

In some embodiments, the drug moiety is selected from the group consisting of a microtubule inhibitor, a DNA cleavage enzyme, an Akt inhibitor, a DNA intercalator, a DNA transcription inhibitor, a DNA cross-linker, and a Dihydrofolate Reductase (DHFR) inhibitor.

In another aspect, provided herein is a method of treating cancer in a patient in need thereof comprising administering to the patient an antibody-based drug conjugate described herein.

In yet another aspect, provided herein is a docking peptide comprising the amino acid sequence of any one of SEQ ID NOs:1-8 or 39-45. In some embodiments, the docking peptide further comprises a lysosome targeting peptide.

In some embodiments, the docking peptide further comprises one or more drug moieties, wherein each of the drug moieties is attached to one of the lysines of the docking peptide. In some embodiments, the one or more drug moieties each further comprises a nuclear localization signal.

In another aspect provided herein are nucleic acids encoding the subject docking peptides, expression vectors and host cells that include such nucleic acids, and methods of making the subject docking peptides.

### BRIEF DESCRIPTION OF THE DRAWINGS

FIG. 1 provides schematics of exemplary alpha (α) helical docking peptides for use in the subject antibody-based drug conjugates provided herein. As shown in FIG. 1, the docking peptides include lysine residues that are strategically arranged in the docking peptide to facilitate the controlled attachment of drug moieties and minimize steric hindrance in the drug conjugate moiety of the subject antibody-based drug conjugate.

FIGS. 2A and 2B provide the amino acid sequence of exemplary alpha helical docking peptides generated by computational modeling and for use in the subject antibody-based drug conjugates provided herein.

FIG. 3 provides a schematic of an exemplary subject antibody-based drug conjugate provided herein. In some embodiments, the subject antibody-based drug conjugate provided herein includes a drug conjugate moiety that is linked to an antibody-based targeting moiety. As discussed herein, the drug conjugate moiety includes an alpha helical docking peptide and drug moieties that are attached to lysine residues positioned at specific, equally spaced locations within the alpha helical docking peptide. The antibody-based targeting moiety includes one or more tumor antigen binding domains (e.g., one or more anti-tumor target antigen scFvs) that allow for targeting of the antibody-based drug conjugate to the tumor microenvironment (TME). In some embodiments, the antibody-based drug conjugate further includes an albumin binding domain (e.g., an anti-human albumin scFv, termed “ABD” in FIG. 3).

FIG. 4 provides an exemplary schematic for making the subject antibody-based drug conjugates.

FIG. 5 provides an overview of the experimental design of a study to assess serum stability of candidate alpha helical peptides generated by computer modeling.

FIGS. 6A and 6B provides the results of studies to assess the serum stability (A) and payload conjugation efficiency (B) of candidate subject alpha helical peptides provided herein.

FIG. 7 provides the results of a study to test the potency of a subject antibody-based drug conjugate with an exemplary antibody-based drug conjugate (“2×Her2 scFv5-Peptide-3×MMAE,” also referred to as “SONP-T3” and “scFv5-Peptide-3×MMAE” herein) on various HER2 positive or HER2 negative cell lines.

FIG. 8 provides an overview of the experimental design of a study to assess the potency of a subject antibody-based drug conjugate with a HER2 positive cell line, BT-474.

FIG. 9 shows the results of the study to assess the potency of a subject antibody-based drug conjugate (“2×Her2 scFv5-Peptide-3×MMAE,” also referred to as “SONP-T3” and “scFv5-Peptide-3×MMAE” herein) on BT-474 cells.

FIG. 10 shows the results of the study to assess the binding of various constructs to human epidermal growth factor receptor 2 (HER2).

FIG. 11 shows the results of a study to evaluate the in vivo antitumor activity of an exemplary antibody-based drug conjugate provided herein (“2×Her2 scFv5-Peptide-3×MMAE,” also referred to as “SONP-T3” and “scFv5-Peptide-3×MMAE” herein) using a BT-474 subcutaneous breast cancer mouse model.

FIGS. 12A-121 provide a summary of a study to assess potential in vivo toxic effects of the various constructs based on hematological profiles.

FIGS. 13A and 13B provide a summary of studies to assess potential in vivo toxic effects of the various constructs based on time course induction of liver aspartate aminotransferase (AST) and alanine aminotransferase (ALT) levels.

FIGS. 14A-C provides a summary of additional studies to assess potential in vivo toxic effects of the various constructs based on creatine toxicity for nephrotoxicity (FIG. 14A); creatine kinase (CK) levels (FIG. 14B), and total bilirubin levels (FIG. 14C).

FIG. 15 provides the results of the assessment of animal weight during the course of the in vivo antitumor activity study.

### DETAILED DESCRIPTION

### A. Overview

Provided herein are novel docking peptides and antibody-based drug conjugates that include such docking peptides. In embodiments, the subject docking peptide allows for the controlled attachment of one or more drug moieties and a tumor targeting moiety that allows for the targeting of the drug moiety to the TME. In some embodiments, the antibody-based drug conjugates include a nuclear localization signal, lysosome targeting peptide and/or albumin binding domain that facilitate delivery of a drug payload into the nucleus of target cells (e.g., tumor target cells). In some embodiments, the antibody-based drug conjugates provided herein advantageously allows for enhanced targeted delivery of a drug payload having a controlled drug-to-antibody ratio (DAR) to a tumor site, thereby improving retention time and drug efficacy. Aspects of the antibody-based drug conjugates are discussed in further detail herein.

### B. Drug Conjugate Moiety

The subject antibody-based drug conjugates include a drug conjugate moiety that is linked to an antibody-based targeting moiety. The drug conjugate moiety includes a docking peptide that allows for the controlled conjugation of drug moieties. Moreover, the docking peptide is small in size (less than 6 kD) and flexible, thereby facilitating enhanced tumor penetration of the antibody-based drug conjugate. Aspects of the drug conjugate moiety are discussed in detail below.

### 1. Docking Peptides

Provided herein are novel alpha helical docking peptides that find use in the subject antibody-based drug conjugates. The docking peptide described herein includes three or more lysine residues that are in the solvent-exposed surface of the peptide and allow for targeted drug moiety conjugation. Such lysine residues allow for efficient and controlled attachment of drug moieties to achieve a desired drug-to-antibody ratio (DAR). Moreover, the alpha-helical docking peptide is small (less than 6 kD) and flexible, thereby allowing for enhanced penetration and delivery to the tumor microenvironment of the subject antibody-based drug conjugate.

In some embodiments, the docking peptide is about 15, about 16, about 17, about 18, about 19, about 20, about 21, about 22, about 23, about 24, or about 25 amino acids in length. In some embodiments, the docking peptide is 26, 27, 28, 29, 30, 31, 32, 33, 34, 35, 36, 37, 38, 39, 40, 41, 42, 43, 44, 45, 46, 47, 48, 49, or 50 amino acids in length.

In certain embodiments, the docking peptide includes 1, 2, 3, 4, 5, 6, 7, 8, 9, or 10 lysine resides for the attachment of drug moieties to the docking peptide. In exemplary embodiments, the lysine residues are separated in the docking peptide by at least 2, 3, 4, 5, 6, 7, 8 or more non-lysine amino acid residues. In some embodiments, the amine side chains of the lysine residues in the docking peptide are separated by at least 10 Å or more in 3D space.

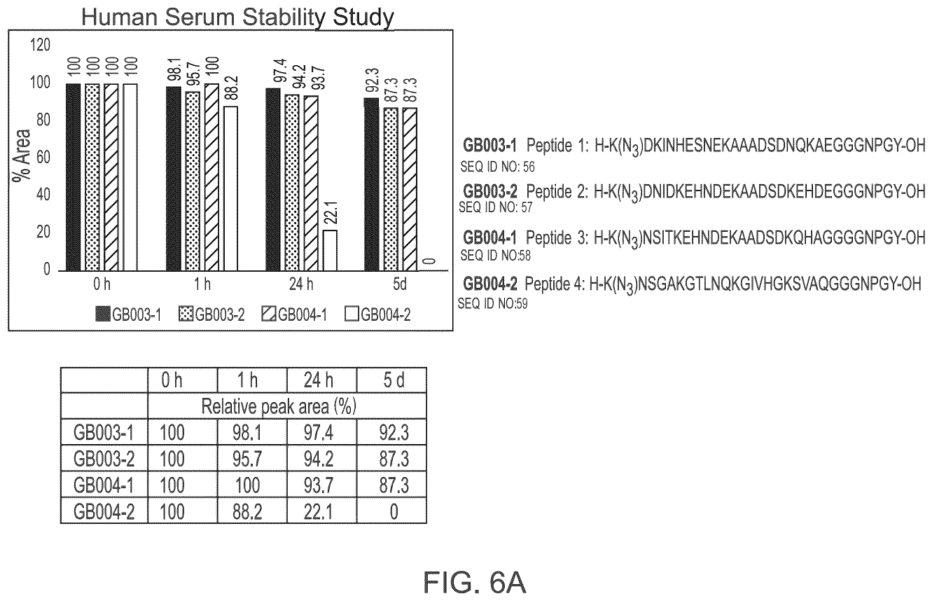

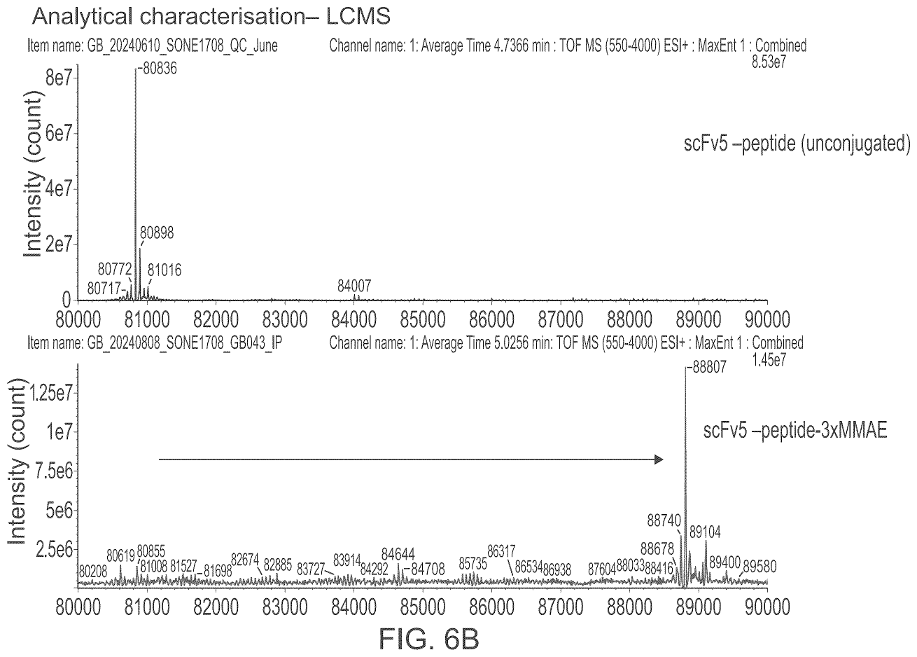

The docking peptides provided herein are selected for low risk of immunogenicity and are highly stable in human serum (see FIG. 6A). In particular, the subject docking peptides provided herein do not include a human MHC class II binding sequence or a known T cell epitope sequence.

In some embodiments, the docking peptides are resistant to lysis by one more or of the following proteases: trypsin, chymotrypsin, metallo-proteinase-2, or metallo-proteinase-9.

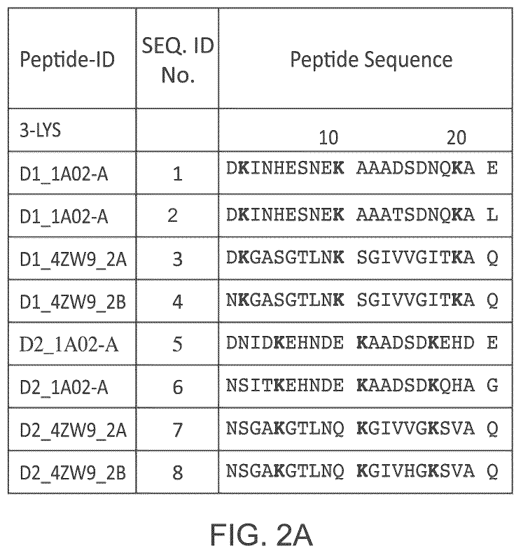

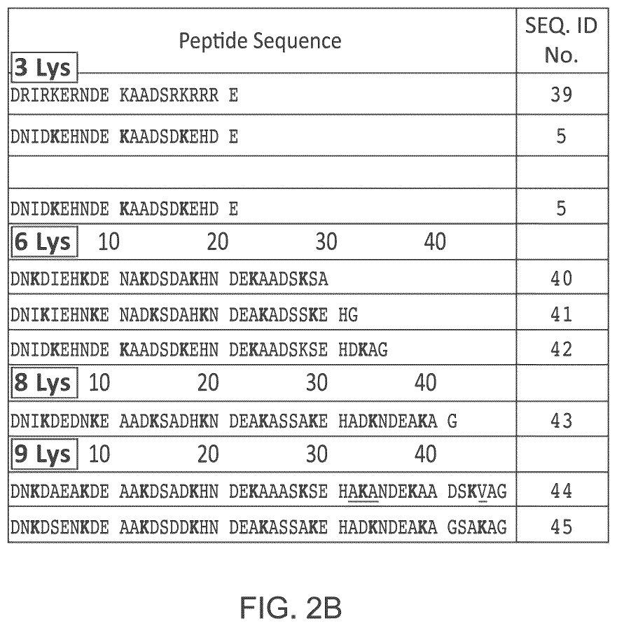

In some embodiments, the docking peptide has an amino acid sequence of one of the sequences set forth in Table 1 or a variant thereof. Additional docking peptides that find use in the subject antibody-based drug conjugates are depicted in FIG. 2B and variants thereof.

TABLE 1

| | SEQ |
|---|---|
| | ID |
| Exemplary Docking Peptide | NO. |
| DKINHESNEKAAADSDNQKAE | 1 |
| DKINHESNSKAAATSDNQKAL | 2 |
| DKGASGTLNKSGIVVGITKAQ | 3 |
| NKGASGTLNKSGIVVGITKAQ | 4 |
| DNIDKEHNDEKAADSDKEHDE | 5 |
| NSITKEHNDEKAADSDKQHAG | 6 |
| NSGAKGTLNQKGIVVGKSVAQ | 7 |
| NSGAKGTLNQKGIVHGKSVAQ | 8 |
| DRIRKERNDEKAADSRKRRRE | 39 |
| DNKDIEHKDENAKDSDAKHNDEKAADSKSA | 40 |
| DNIKIEHNKENADKSDAHKNDEAKADSSKEHG | 41 |
| DNIDKEHNDEKAADSDKEHNDEKAADSKSEHDKAG | 42 |
| DNIKDEDNKEAADKSADHKNDEAKASSAKEHADKNDEAKAG | 43 |
| DNKDAEAKDEAAKDSADKHNDEKAAASKSEHAKANDEKAADS | 44 |
| KVAG | |
| DNKDSENKDEAAKDSDDKHNDEAKASSAKEHADKNDEAKAGS | 45 |
| AKAG | |

In some embodiments, the docking peptide includes 1, 2, 3, 4, 5 or more of the same docking peptides in Table 1 or FIG. 2 or a variant thereof.

In some embodiments, the docking peptide is a variant docking peptide having a sequence that includes 1, 2, 3, 4, 5, 6, 7, 8, 9 or 10 amino acid substitutions compared to a docking peptide sequence of Table 1 or FIG. 2. In some embodiments, the variant docking peptide has 1, 2, 3, 4, 5, 6, 7, 8, or 9 lysine resides for the attachment of drug moieties. In some embodiments, the variant docking peptide includes an amino acid substitution of one or more of its lysine for an amino acid that facilitates conjugation to a particular payload of interest. In some embodiments, the docking peptide is a variant of a docking peptide in Table 1 or FIG. 2 that includes a substitution of 3, 6, 8, or 9 lysine residues for an amino acid that facilitates conjugation to a particular payload of interest. In some embodiments, the lysine residues are substituted for an amino acid that is not included elsewhere the docking peptide. In some embodiments, the docking peptide is a variant of a docking peptide in Table 1 or FIG. 2 wherein 1, 2, 3, 4, 5, 6, 7, 8 or 9 of the lysine residues are substituted from a cysteine residue. In some embodiments, the docking peptide includes a combination of lysine and cysteine residues that allow for conjugation of different drug moieties on the same docking peptide. Linkers for conjugating In exemplary embodiments, the docking peptide is a variant of SEQ ID NO: 41, 42, 43, wherein 3 of the lysine residues are substituted for three cysteine residues.

In some embodiments, the antibody-based drug conjugate includes two or more docking peptides, each docking peptide allowing for attachment of a different drug moiety.

To enhance trafficking of the antibody-based drug conjugate to the tumor microenvironment, the docking peptide provided herein may include further a lysosome targeting peptide. In some embodiments, the lysosome targeting peptide is attached to the N-terminus of the docketing peptide. In other embodiments, the lysosome targeting peptide is attached to the C-terminus of the docking peptide. In some embodiments, the antibody-based drug conjugate includes a peptide signal that facilitates clathrin-mediated endocytosis, caveolae-mediated endocytosis and/or micropinocytosis. In exemplary embodiments, the antibody-based drug conjugate includes one of the lysosome targeting peptide sequences in Table 2. In exemplary embodiments, the lysosome targeting peptide does not include a lysine. Additional lysosome targeting peptide sequences are included and disclosed in Bonifacino and Traub, Annu Rev Biochem 72:395-447 (2003), which is incorporated by reference in its entirety and particularly for disclosures related to lysosome and endosome targeting sequences. In some embodiments, the lysosome targeting peptide is attached to the N-terminus of the docking peptide. In other embodiments, the lysosome targeting peptide is attached to the C-terminus of the docking peptide.

TABLE 2

| Lysosome Targeting Peptide Sequences | SEQ ID NO. |
|---|---|
| NPXY, wherein X is any amino acid | 9 |
| NPGY | 10 |
| YXXØ, wherein X is any amino acid and Ø is an amino | 11 |
| acid with a bulky hydrophobic side chain | |
| [DE]XXXL[LI], wherein X is any amino acid | 12 |
| DXXLL, X is any amino acid | 13 |

In addition to the subject alpha-helical docking peptides, also provided herein are nucleic acids encoding the docking peptide, expression vectors and host cells for making the docking peptide. The docking peptides can be made according to any suitable technique known in the art, including standard molecular biology and recombinant protein expression techniques known in the art.

### 2. Drug Moieties

The antibody-based drug conjugates provided herein can be conjugated to one or more drug moieties. The one or more drug moieties are attached to the docking peptide using a suitable linker (e.g., a cleavable linker). In some embodiments, the docking peptide is conjugated to 2, 3, 4, 5, 6, 7, 8, 9, or 10 or more drug moieties. The drug moieties of the antibody-based drug conjugate can be any number of agents, including, but not limited to cytotoxic agents such as chemotherapeutic agents, growth inhibitory agents, toxins (for example, an enzymatically active toxin of bacterial, fungal, plant, or animal origin, or fragments thereof), or a radioactive isotope (that is, a radioconjugate). Any suitable drug payload can be included, in the antibody-based drug conjugate. In exemplary embodiments, the drug moiety is a cytotoxic or cytostatic agent that is detrimental to cells.

In some embodiments, particularly for antibody-based drug conjugate used for the treatment of a cancer or other medical applications, the drug can be an antiproliferative and/or cytotoxic drug or other types of compounds or biological small peptide. Such drugs include, in general, DNA damaging agents, anti-metabolites, natural products and their analogs. Exemplary classes of cytotoxic agents include the enzyme inhibitors such as dihydrofolate reductase (DHFR) inhibitors, and thymidylate synthase inhibitors, DNA intercalators, DNA cleavers (e.g., Bleomycin A2 and Calicheamicin y-1), topoisomerase inhibitors, protein kinase b (Akt) inhibitors (e.g., GDC-0068), the anthracycline family of drugs, the vinca drugs, the mitomycins, the bleomycins, the cytotoxic nucleosides, the pteridine family of drugs, diynenes, the podophyllotoxins, dolastatins, maytansinoids, differentiation inducers, and taxols.

In certain embodiments, the antibody-based drug conjugate includes one of the following: methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, caminomycin, aminopterin, tallysomycin, podophyllotoxin and podophyllotoxin derivatives such as etoposide or etoposide phosphate, vinblastine, vincristine, vindesine, taxanes including taxol, taxotere retinoic acid, butyric acid, N8-acetyl spermidine, camptothecin, calicheamicin, esperamicin, ene-diynes, duocarmycin A, duocarmycin SA, calicheamicin, camptothecin, maytansinoids (including DM1), monomethylauristatin E (MMAE), monomethylauristatin F (MMAF), and maytansinoids (DM4) and their analogues.

In some embodiments, the antibody-based drug conjugate includes one or more microtubule inhibitors. Microtubule inhibitors stabilize or destabilize microtubules, thereby suppressing microtubule dynamics required for proper mitotic function, effectively blocking cell cycle progression, and resulting in apoptosis. Exemplary microtubule inhibitors include, but are not limited to: dolastatin-10, auristatins (MMAE, MMAF, PE), maytansines (DM-1, DM-4, >10 derivatives), vinorelbine, paclitaxel, epothilone B, and tubulysins (IM-2, B).

In some embodiments, the antibody-based drug conjugate includes one or more DNA cleavers. DNA cleavers work by introducing strand breaks in DNA. Exemplary DNA cleavers include, but are not limited to, bleomycin A2 and calicheamicin γ-1.

In some embodiments, the antibody-based drug conjugate includes one or more protein kinase B (Akt) inhibitors. Akt is a protein kinase that plays a key role in multiple cellular processes such as apoptosis, cell proliferation, glucose metabolism, transcription, and cell migration. Inhibition can lead to the induction of apoptosis. In some embodiments, the Akt inhibitor is GDC-0068.

In some embodiments, the antibody-based drug conjugate includes one or more DNA intercalators. DNA intercalators fit between nucleic acid base pairs, thereby preventing DNA replication. Exemplary DNA intercalators include, but are not limited to: doxorubicin hydrochloride, epirubicin hydrochloride, PNU-159682, duocarmycins, PBD dimers, oligomycin C, daunorubicin hydrochloride, valrubicinm, topotecan and desmethyl-topotecan.

In some embodiments, the antibody-based drug conjugate includes one or more DNA transcription inhibitors. In exemplary embodiments, the transcription inhibitor is dactinomycin.

In certain embodiments, the antibody-based drug conjugate includes one or more DNA cross-linkers. In some embodiments, the DNA cross-linker is mitomycin C.

In exemplary embodiments, the antibody-based drug conjugate includes one or more dihyrofolate reductase (DHFR) inhibitors. DHFR has a critical role in regulating the amount of tetrahydrofolate in the cell. Tetrahydofolate and its derivatives are essential for purine and thymidine synthesis, which are important for cell proliferation and cell growth. In some embodiments, the DHFR inhibitor is methotrexate.

In certain embodiments, the drug included in the antibody-based drug conjugate is a toxin. Toxins may be used as antibody-toxin conjugates and include bacterial toxins such as diphtheria toxin, Pseudomonas aeruginosa endotoxin, plant toxins such as ricin, small molecule toxins such as geldanamycin (Mandler et al., J. Nat. Cancer Inst. 92 (19):1573-1581 (2000), Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). Toxins may exert their cytotoxic and cytostatic effects by mechanisms including tubulin binding, DNA binding, or topoisomerase inhibition.

In some embodiments, each of the drug moieties is further attached to a nuclear localization signal (NLS) peptide that facilitates the trafficking of the drug moiety of the antibody-based drug conjugate to the nucleus of a target cell (e.g., a tumor cell). Any suitable nuclear localization signal capable of facilitating the transport of the drug moiety to the nucleus of a target cell may be included in the docking peptide. Exemplary nuclear localization signals that can be included in the subject docking peptides are listed in Table 3. In exemplary embodiments, the nuclear localization signal does not include a lysine, e.g., SEQ ID 19, 20 or 23.

TABLE 3

| | SEQ |
|---|---|
| | ID |
| Nuclear Localization Signals | NO. |
| PKKKRKV | 14 |
| KRPAATKKAGQAKKKK | 15 |
| KRPMNAFIVWSRDQRRK | 16 |
| RPRRK | 17 |
| SSNFGPMKGGNRFFRSSGPY | 18 |
| RSGGNHRRNGRGGRGGYNRRNNGYHPY | 19 |
| LRLTLLELVRRLNGNG | 20 |
| GKISKHWTGI | 21 |
| SKKSLED | 22 |
| PPRSKKRI | 55 |
| RQARRNRRRRWR | 23 |
| GRKKRRQRRRAP | 24 |
| MPKTRRRPRRSQRKRPPT | 25 |
| KSAKISKPLH-(X)₂₈-KNKEISMP, wherein X is any | 26 |
| amino acid | |
| KVTKNKS-(X)₆-KRRGKPGP, wherein X is any amino | 27 |
| acid | |
| TAKRS-(X)₃₄-KKKGSKTS, wherein X is any amino | 28 |
| acid | |
| GYGPKKKRKVGG | 29 |

a. Maytansinoids

In some embodiments, the antibody-based drug conjugate includes a maytansinoid. Maytansine compounds suitable for use as maytansinoid drug moieties are well known in the art, and can be isolated from natural sources according to known methods, produced using genetic engineering techniques (see Yu et al., PNAS 99:7968-7973 (2002)), or maytansinol and maytansinol analogues prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides) and those having modifications at other positions.

Exemplary maytansinoid drug moieties also include those having modifications such as: C-9-SH (U.S. Pat. No. 4,424,219) (prepared by the reaction of maytansinol with H2S or P2S5); C-14-alkoxymethyl (demethoxy/CH2OR) (U.S. Pat. No. 4,331,598); C-14-hydroxymethyl or acyloxymethyl (CH2OH or CH2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Of particular use are DM1 (disclosed in U.S. Pat. No. 5,208,020, incorporated by reference) and DM4 (disclosed in U.S. Pat. No. 7,276,497, incorporated by reference). See also a number of additional maytansinoid derivatives and methods in U.S. Pat. No. 5,416,064, WO/01/24763, U.S. Pat. Nos. 7,303,749, 7,601,354, U.S. Ser. No. 12/631,508, WO02/098883, U.S. Pat. Nos. 6,441,163, 7,368,565, WO02/16368 and WO04/1033272, all of which are expressly incorporated by reference in their entirety.

b. Auristatins and Dolastatins

In some embodiments, the antibody-based drug conjugate comprises a dolastatin or a dolastatin peptide analog and derivative, or an auristatin (U.S. Pat. Nos. 5,635,483; 5,780,588), auristatin analog or derivative. Dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptide drug moiety (WO 02/088172).

Exemplary auristatin embodiments include the N-terminus linked monomethylauristatin drug moieties DE and DF, disclosed in “Senter et al, Proceedings of the American Association for Cancer Research, Volume 45, Abstract Number 623, presented Mar. 28, 2004 and described in United States Patent Publication No. 2005/0238648, the disclosure of which is expressly incorporated by reference in its entirety.

An exemplary auristatin embodiment is MMAE (see U.S. Pat. No. 6,884,869 expressly incorporated by reference in its entirety).

Another exemplary auristatin embodiment is MMAF (see US 2005/0238649, U.S. Pat. Nos. 5,767,237 and 6,124,431, expressly incorporated by reference in their entirety).

c. Calicheamicin

In some embodiments, the antibody-based drug conjugate comprises one or more calicheamicin or calicheamicin derivative or analog. For example, Mylotarg is the first commercial ADC drug and utilizes calicheamicin γ1 as the payload (see U.S. Pat. No. 4,970,198, incorporated by reference in its entirety). Additional calicheamicin derivatives are described in U.S. Pat. Nos. 5,264,586, 5,384,412, 5,550,246, 5,739,116, 5,773,001, 5,767,285 and 5,877,296, all expressly incorporated by reference. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ1I, α2I, α2I, N-acetyl-γ1I, PSAG and θ1I (Hinman et al., Cancer Research 53:3336-3342 (1993), Lode et al., Cancer Research 58:2925-2928 (1998) and the aforementioned U.S. patents to American Cyanamid). Another anti-tumor drug that the antibody-based drug conjugate can be conjugated is QFA which is an antifolate. Both calicheamicin and QFA have intracellular sites of action and do not readily cross the plasma membrane. Therefore, cellular uptake of these agents through albumin binding domain mediated internalization greatly enhances their cytotoxic effects.

d. Duocarmycins

In some embodiments, the antibody-based drug conjugate comprises one or more duocarmycin or duocarmycin derivative or analog. CC-1065 (see U.S. Pat. No. 4,169,888, incorporated by reference) and duocarmycins are members of a family of antitumor antibiotics utilized in ADCs. These antibiotics appear to work through selectively alkylating DNA sequences at the N3 of adenine in the minor groove, which initiates a cascade of events that result in apoptosis.

Important members of the duocarmycins include duocarmycin A (U.S. Pat. No. 4,923,990, incorporated by reference) and duocarmycin SA (U.S. Pat. No. 5,101,038, incorporated by reference), and a large number of analogues as described in U.S. Pat. Nos. 7,517,903, 7,691,962, 5,101,038; 5,641,780; 5,187,186; 5,070,092; 5,070,092; 5,641,780; 5,101,038; 5,084,468, 5,475,092, 5,585,499, 5,846,545, WO2007/089149, WO2009/017394A1, 5,703,080, 6,989,452, 7,087,600, 7,129,261, 7,498,302, and 7,507,420, all of which are expressly incorporated by reference.

e. Other Cytotoxic Agents

Other antitumor agents that can be included in the drug payload of the antibody-based drug conjugates provided herein include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296). Further, the antitumor agent can also include a tubulysin, a topoisomerase inhibitor, a DNA-alkylator, or an RNA polymerase II inhibitor.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993.

In some embodiments, the subject antibody-based drug conjugate includes a drug with nucleolytic activity (e.g., a ribonuclease or a DNA endonuclease such as a deoxyribonuclease; DNase).

For selective destruction of the tumor, the subject antibody-based drug conjugate may include a highly radioactive atom. A variety of radioactive isotopes are available for the production of radioconjugated antibodies. Examples include At211, I131, I125, Y90, Re186, Re188, Sm153, Bi212, P32, Pb212 and radioactive isotopes of Lu.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as Tc99m or 1123, Re186, Re188 and In111 can be attached via a cysteine residue in the peptide. Yttrium-90 can be attached via a lysine residue. The IODOGEN method (Fraker et al (1978) Biochem. Biophys. Res. Commun. 80:49-57 can be used to incorporate Iodine-123. “Monoclonal Antibodies in Immunoscintigraphy” (Chatal, CRC Press 1989) describes other methods in detail.

### 3. Drug Loading

Drug loading is represented by p and is the average number of drug moieties per docking peptide in a molecule. Drug loading (“p”) may be 1, 2, 3, 4, 5, 6, 7, 8, 9, 10, 11, 12, 13, 14, 15, 16, 17, 18, 19, 20 or more moieties (D) per docking peptide, although frequently the average number is a fraction or a decimal. Generally, drug loading of from 1 to 4 is frequently useful, and from 1 to 2 is also useful. The antibody-based drug conjugates provided herein include collections of docking peptides conjugated with a range of drug moieties, from 1 to 20. The average number of drug moieties per docking peptides in preparations of the antibody-based drug conjugates from conjugation reactions may be characterized by conventional means such as mass spectroscopy and, ELISA assay.

The quantitative distribution of antibody-based drug conjugates in terms of p may also be determined. In some instances, separation, purification, and characterization of homogeneous antibody-based drug conjugates where p is a certain value from antibody-based drug conjugates with other drug loadings may be achieved by means such as electrophoresis.

For some antibody-based drug conjugates, p may be limited by the number of attachment sites on the docking peptide. For example, where the attachment is a lysine residue, as in the exemplary embodiments above, a docking peptide may have only one or several lysine residues through which a linker may be attached. In certain embodiments, higher drug loading, e.g., p>5, may cause aggregation, insolubility, toxicity, or loss of cellular permeability of certain antibody-drug conjugates. In certain embodiments, the drug loading for an antibody-based drug conjugate of the invention ranges from 1 to about 8; from about 2 to about 6; from about 3 to about 5; from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7. Indeed, it has been shown that for certain ADCs, the optimal ratio of drug moieties per antibody may be less than 8, and may be about 2 to about 5. See US 2005-0238649 A1 (herein incorporated by reference in its entirety).

The loading (drug/docking peptide ratio) of an antibody-based drug conjugate may be controlled in different ways, e.g., by: (i) limiting the molar excess of drug-linker intermediate or linker reagent relative to albumin binding domain/target antigen binding domain, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments (such as thioMab or thioFab prepared as disclosed herein and in WO2006/034488 (herein incorporated by reference in its entirety)).

It is to be understood that where more than one nucleophilic group reacts with a drug-linker intermediate or linker reagent followed by drug moiety reagent, then the resulting product is a mixture of antibody-based drug conjugates with a distribution of one or more drug moieties attached to a docking peptide. The average number of drugs per docking peptide may be calculated from the mixture by a dual ELISA antibody assay, which is specific for the docking peptide and specific for the drug. Individual antibody-based drug conjugate molecules may be identified in the mixture by mass spectroscopy and separated by HPLC, e.g., hydrophobic interaction chromatography.

In some embodiments, a homogeneous antibody-based drug conjugate with a single loading value may be isolated from the conjugation mixture by electrophoresis or chromatography.

### C. Linkers

Components of the antibody-based drug conjugates provided herein (e.g., targeting moieties, docking peptides, drug moieties) are attached to each other using linkers. Any suitable linker can be used. Suitable linkers include, but are not limited to, hydrazones, disulfides, peptides and thioether bond linkers.

Linkers are used, for example, for attaching drug moieties to the docking peptide and the docking peptide to the antigen-based targeting moiety. Linkers can also be used to attach targeting moieties and/or albumin binding domains in embodiments wherein such components are included. In exemplary embodiments, the linker does not include a lysine.

Preferably, the linker is stable at systemically relevant pHs for multiple days to allow the antibody-based drug conjugate to localize to the tumor microenvironment. The stability further prevents the premature release of the drug moiety, which would indiscriminately damage non-cancer cells and tissues, thereby lowering the therapeutic index of the antibody-based drug conjugates. Upon internalization by the target cell, the subject antibody-based drug conjugates are capable of releasing the drug moiety.

In preferred embodiments, the linker utilizes the ε-amino group of lysine. In some embodiments, the attachment of the drug moiety to the docking peptide is via a lysine residue on the docking peptide and/or drug moiety. In certain embodiments, the linker is 4-(4-acetylphenoxy) butanoic acid (AcBut). See, e.g., DiJoseph et al., Hematol Meet Rep. 2:74-7 (2008). In certain embodiments, the linker is attached to a naturally or engineered cysteine residue of the targeting polypeptide. Exemplary cysteine linkers include, but are not limited to, maleimidocaproyl (mc), maleimidomethyl cyclohexane-1-carboxylate (mmc), valine-citrulline (vc), valine-alanine (va), mcc-triazole spacer-PEG7-x-lysine-PABC-SN-38, and maleimidomethyl cyclohexane-1-carboxyl hydrazide.

Linkers used in the subject antibody-based drug conjugate can be cleavable or non-cleavable or combinations thereof. A cleavable linker may include a disulfide bond, acid-cleavable linkage, ester bond, anhydride bond, biodegradable bond, or enzyme-cleavable linkage. A non-cleavable linker may include an amide or phosphate bond.

In some embodiments, the drug moiety is attached to the docking peptide by a cleavable linker. The cleavable linkers provided herein are cleavable under intracellular or extracellular conditions. Preferably, cleavable linkers used in the antibody-based drug conjugates herein are stable in blood circulation for a long period of time and efficiently release their drug payload in the target cell or target microenvironment. In some embodiments, the linker is cleavable by a cleaving agent that is present in the intracellular environment (for example, within a lysosome or endosome or caveolae). Cleavable linkers can undergo cleavage using three commonly used mechanisms: 1) protease-sensitivity; 2) pH-sensitivity; and 3) glutathione-sensitivity. In some embodiments, the linker is a protease-sensitive linker that is cleaved by a dominant protease found in the target cell. Exemplary protease-sensitive linkers include, for example, valine-citrulline (vc) dipeptide that is cleaved by intracellular cathepsin B. See, e.g., Dubowchik and Firesone et al., Bioconjug Chem. 13 (4):855-69 (2002). Cathepsin B has an optimum pH of about 4.5 to 5.5 and no activity at neutral pH. In some embodiments, the cleavable linker is a tetrapeptide cleavable linker (e.g., a gly-gly-phe-gly linker).

In other embodiments, the cleavable linker is pH-sensitive, that is, sensitive to hydrolysis at certain pH values. Typically, the pH-sensitive linker is hydrolyzable under acidic conditions. In certain embodiments, the linker is an acid-sensitive linker that takes advantage of the lower pH in the endosomal (pH=5-6) and lysosomal (pH=4.8) cellular compartments, as compared to the cytosol (pH=7.4), to trigger hydrolysis of an acid labile group within the linker (e.g., hydrazone). See, e.g., Ritchie et al., MAbs 5 (1):13-21 (2013).

In other embodiments, the linker is a glutathione-sensitive linker that exploits the higher concentrations of intracellular glutathione as compared to that in plasma. Such linkers include a disulfide bridge that releases the cytotoxin upon reduction by intracellular glutathione.

In yet other embodiments, the linker is cleavable under reducing conditions (for example, a disulfide linker). A variety of disulfide linkers are known in the art, including, for example, those that can be formed using SATA (N-succinimidyl-5-acetylthioacetate), SPDP (N-succinimidyl-3-(2-pyridyldithio) propionate), SPDB (N-succinimidyl-3-(2-pyridyldithio) butyrate) and SMPT (N-succinimidyl-oxycarbonyl-alpha-methyl-alpha-(2-pyridyl-dithio) toluene)-, SPDB and SMPT. (See, e.g., Thorpe et al., Cancer Res. 47:5924-5931 (1987); Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, the linker is an SMCC (succinimidyl 4-(N-maleimidomethyl)cyclohexane-1-carboxylate) linker. SMCC is a hetero-bifunctional crosslinker that contain N-hydroxysuccinimide (NHS) ester and maleimide groups that allow covalent conjugation of amine- and sulfhydryl-containing molecules.

In some embodiments, the cleavable linker includes a nuclear localization signal that is exposed upon cleavage of the cleavable linker. In preferred embodiments, cleavage of the cleavable linker results in a drug moiety with an exposed nuclear localization signal that facilitates the trafficking of the drug moiety to the nucleus of a target cell (e.g., a tumor cell). Suitable nuclear localization signals can be included in the cleavable linker are listed in Table 3 herein.

In certain embodiments, the components are attached using site-specific conjugation. Techniques for site specific conjugation include but are not limited to 1) genetic engineering of cysteine or seleno cysteine residues, 2) incorporation of non-natural amino acids (nnAA) possessing reactive handles (by either genetic engineering or enzymatic modification) and 3) enzymatic modification. See, e.g., Panowski et al., MAbs 6 (1):34-45 (2014).

In certain embodiments, the linker is non-cleavable. Such antibody-based drug conjugates rely on the degradation of the targeting polypeptide for release of the drug. Exemplary non-cleavable linkers include thioether linkers used in T-DM1 and maleimidocaproic acid linked to monomethyl auristatin F (mc-MMAF), used in some ADCs under clinical evaluation, such as depatuxizumab mafodotin.

In exemplary embodiments, the antibody-based drug conjugate includes one or more polypeptide linkers. Such polypeptide linkers can be used to attach the targeting moiety and docking peptide of the antibody-based drug conjugate and/or the drug moiety and docking peptide. In some embodiments, the polypeptide linker is a gly-ser linker that includes glycine and serine residues. Exemplary gly-ser linkers include, for example, (GS)n, (GSGGS)n, (GGGGS)n, and (GGGS)n, where n is an integer (e.g., 1 to 10) of at least one (and generally from 3 to 4), glycine-alanine polymers, alanine-serine polymers, and other flexible linkers.

In certain embodiments, the polypeptide linker may include other polypeptides in addition to the gly-ser linker. For example, the gly-ser linker may be inserted between two other sequences of the polypeptide linker. In other embodiments, a gly-ser linker is attached at one or both ends of another sequence of the polypeptide linker. In yet other embodiments, two or more gly-ser linker are incorporated in series in a polypeptide linker.

In certain embodiments, the polypeptide linker is at least 5, 10, 15, 20, 25, 30, or 35 amino acids in length. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Exemplary polypeptide linkers that can be used with the subject antibody-based drug conjugates are included in Table 4. Additional polypeptide linkers are disclosed, for example, in U.S. Pat. Nos. 5,525,491 and 6,660,843, which are incorporated by reference in their entirety, particularly for teachings related to polypeptide linkers, as well as the Linker Database (ibi.vu.nl/programs/linkerdbwww).

TABLE 4

| | | SEQ |
|---|---|---|
| Name | Sequence | ID NO: |
| (GGGGS)₁ | GGGGS | 30 |
| or GGGGS | | |
| (GGGGS)₂ | GGGGGGGGS | 31 |
| (GGGGS)₃ | GGGGSGGGGSGGGGS | 32 |
| (GGGGS)₄ | GGGGSGGGGSGGGGSGGGGS | 33 |
| (GGGGS)₅ | GGGGSGGGGSGGGGSGGGGSGGGGS | 34 |
| (GGGGS)₆ | GGGGSGGGGSGGGGSGGGGSGGGGSGGGGS | 35 |
| (GGGGS)₇ | GGGGSGGGGSGGGGSGGGGSGGGGSGGGGSGG | 36 |
| | GGS | |
| (GKPGS)₅ | GKPGSGKPGSGKPGSGKPGSGKPGS | 37 |
| (GKPGS)₆ | GKPGSGKPGSGKPGSGKPGSGKPGSGKPGS | 38 |

Polypeptide linkers can be introduced into polypeptide sequences using techniques known in the art. Modifications can be confirmed by DNA sequence analysis. Plasmid DNA can be used to transform host cells for stable production of the translated polypeptides.

Alternatively, a variety of nonproteinaceous polymers can used in the subject drug/albumin conjugates, including but not limited to polyethylene glycol (PEG), polypropylene glycol.

Often the linker is not substantially sensitive to the extracellular environment. As used herein, “not substantially sensitive to the extracellular environment,” in the context of a linker, means that no more than about 20%, 15%, 10%, 5%, 3%, or no more than about 1% of the linkers, in a sample antibody-based drug conjugate, are cleaved when the antibody-based drug conjugate is present in an extracellular environment (for example, in plasma).

Whether a linker is not substantially sensitive to the extracellular environment can be determined, for example, by incubating the antibody-drug conjugate compound with plasma or serum for a predetermined time period (for example, 2, 4, 8, 16, or 24 hours) and then quantitating the amount of free drug present in the plasma.

In other, non-mutually exclusive embodiments, the linker promotes cellular internalization. In certain embodiments, the linker promotes cellular internalization when conjugated to the therapeutic agent (that is, in the milieu of the linker-therapeutic agent moiety of the antibody-drug conjugate compound as described herein). In yet other embodiments, the linker promotes cellular internalization when conjugated to both the drug and the targeting polypeptide of the conjugate.

### D. Antibody-Based Targeting Moiety

In some embodiments, the antibody-based drug conjugate provided herein includes one or more antibody-based targeting moieties that facilitates the targeting of the antibody-based drug conjugate to a particular target site (e.g., a tumor cell). The targeting moiety includes, for example, one or more antibody-based binding domains, wherein an immunoglobulin variable heavy domain (VH) and variable light domain (VL) form an antigen binding domain that binds an antigen of the target cell of interest. Exemplary antibody based targeting moieties include but are not limited to: a humanized antibody or binding fragment thereof, a chimeric antibody or binding fragment thereof, a monoclonal antibody or binding fragment thereof, a bispecific antibody or binding fragment thereof, a monovalent Fab′, a divalent Fab2, a single-chain variable fragment (scFv), a diabody, a minibody, nanobody, single-domain antibody (sdAb), or camelid antibody or binding fragment thereof. In exemplary embodiments, the targeting moiety is an scFv that binds a target cell antigen with high specificity and high affinity. Preferably the targeting scFv binds an antigen that is high expressed on the target cells (e.g., tumor cells) with minimal expression on normal cells.

In some embodiments, the antibody-based drug conjugate includes two or more antigen binding domains that each bind to the same target antigen. In some embodiments, the antibody-based drug conjugate includes two or more antigen binding domains that each bind to a different target antigen. In some embodiments, the antibody-based drug conjugate includes one anti-target antigen scFv. In other embodiments, the antibody-based drug conjugate includes two anti-target antigen scFvs. In some embodiments, the antibody-based drug conjugate includes 2, 3, 4, 5, 6, 7, 8, 9, or 10 anti-target antigen scFvs. In some embodiments, each of the anti-target antigen scFvs included in the antibody-based drug conjugate binds the same antigen. In some embodiments, the anti-target antigen scFvs included in the antibody-based drug conjugate binds to the same target antigen with different affinities. In other embodiments, each of the anti-target antigen scFvs included in the antibody-based drug conjugate binds a different target antigen.

### 1. Tumor Target Antigens

In some embodiments, the antibody-based anti-target antigen moieties of the antibody-based drug conjugate are anti-tumor antigen moieties. In exemplary embodiments, the anti-tumor antigen moieties are anti-tumor antigen scFvs. Such scFvs bind to tumor antigen that are expressed at a higher level on tumor cells as compared to normal cells. Suitable tumor antigens include, for example, CD33 (acute myeloid leukemia), CD30 (Hodgkin's lymphoma and systemic anaplastic large cell lymphoma), HER2 (HER2-positive breast cancer), CD22 (aggressive/follicular non-Hodgkin's lymphoma, acute lymphoblastic leukemia, and diffuse large B-cell lymphoma (DLBCL)), CD79b (DLBCL and follicular non-Hodgkin's lymphoma), NaPi2b (non-small-cell lung cancer and ovarian tumor), glycoprotein NMB (breast cancer and melanoma), CD19 (DLBCL and acute lymphoblastic leukemia), CD138 (multiple myeloma), PSMA (prostate cancer), CEA (colorectal cancer), guanlyl cyclase C (gastrointestinal tumor), CD198 (acute myeloid leukemia), EGFR (glioblastoma, non-small-cell lung cancer, and squamous cell tumors), CD52 (chronic lymphocytic leukemia), and CD74 (chronic lymphocytic leukemia, multiple myeloma, and non-Hodgkin's lymphoma). Additional tumor antigens include, but are not limited to, FOLR1 (ovarian cancer), CD37 (non-Hodgkin's lymphoma), mesothelin (mesothelin expressing tumors), CECAMS (solid tumors), LAMP1 (solid tumors), and GPNMB (melanoma). Additional target antigens include, but are not limited to: CD56, TROP2, Mucin 1 (Sialogycotope CA6), STEAP1 (prostate cancer), Mesotehlin, Nectin 4, ENP3, guanylyl cyclase C (GCC), SLC44A4, NaPi2b, CD70 (TNFSF7), Cap, 5T4, SLTRK6, SC-16, LIV-1 (ZIP6), and P-Cadherin.

In certain embodiments, the antibody-based drug conjugate includes one or more anti-tumor antigen scFvs that include the six CDRs (vhCDR1-3 and vlCDR1-3) or the variable heavy domain and variable light domain of one of the following antibodies: alemtuzumab (anti-CD52), trastuzumab (anti-HER2), ibritumomab (anti-CD20), brentuximab (anti-CD30), bevacizumab (anti-VEGF), cetuximab (anti-EGFR), glemtumamab (anti-GPNMB), polatuzumab (anti-CD79b), denintuzumab (anti-CD19), mirvetuximab (anti-FOLR1), coltuximab (anti-CD19), naratuximab (anti-CD37), indatuximab (anti-CD138), lorvotuzumab (anti-CD56), Sacituzumab (anti-TACSTD2), labetuzumab (anti-CECAM5), mirvetuximab (anti-folate receptor-alpha), vandortuzumab (anti-STEAP1), and anetumab (anti-mesothelin).

In some embodiments, the antibody-based drug conjugate includes one or more anti-tumor antigen scFvs that include a variable heavy domain and a variable light domain that are variants of the variable heavy domain and variable light domain of one of the following antibodies: alemtuzumab (anti-CD52), trastuzumab (anti-HER2), ibritumomab (anti-CD20), brentuximab (anti-CD30), bevacizumab (anti-VEGF), cetuximab (anti-EGFR), glemtumamab (anti-GPNMB), polatuzumab (anti-CD79b), denintuzumab (anti-CD19), mirvetuximab (anti-FOLR1), coltuximab (anti-CD19), naratuximab (anti-CD37), indatuximab (anti-CD138), lorvotuzumab (anti-CD56), Sacituzumab (anti-TACSTD2), labetuzumab (anti-CECAM5), mirvetuximab (anti-folate receptor-alpha), vandortuzumab (anti-STEAP1), and anetumab (anti-mesothelin). In some embodiments, the variant includes from 1, 2, 3, 4, 5, 6, 7, 9, 10, amino acid changes compared to the VH and/or VL domain of the aforementioned antibodies. In some embodiments, the amino acid change(s) are located in one or more CDRs (i.e., vhCDR1-3 and vlCDR1-3). In certain embodiments, the amino acid changes are in a framework region. In exemplary embodiments, the anti-tumor antigen scFv variant is capable of binding to a tumor antigen as measured by at least one of a surface plasmon resonance (SPR) and/or BLI (biolayer interferometry, e.g., Octet assay) assay.

Exemplary tumor antigen binding domains are disclosed, for example in U.S. Pat. Nos. 5,846,534, 6,627,196, 7,371,379, 5,776,456, 5,736,137, 7,090,843, 6,217,866, 8,691,531, 9,637,547, 9,221,914, 9,023,351, US20150147317, US20130058948, and US20210196835, which are incorporated by reference, particular in pertinent parts relating to ant-tumor antigen antibodies, CDRs (vhCDR1-3 and vlCDR1-3) and VH/VL tumor antigen binding domain components.

### E. Albumin Binding Domain (ABD)

In some embodiments, the antibody-based drug conjugates provided herein include an albumin binding domain. Antibody-based drug conjugates that include an albumin binding domain (ABD) can bind human serum albumin (SA), which allows the fusion protein to be taken in by cells by macropinocytosis. In certain embodiments, the ABDs described herein bind at a pH range of about pH 5.5 to about pH 7.2. In the early endosome, such SA bound ABD fusion proteins bind to FcRn via SA at an acidic pH (e.g., pH 5.5), which in turn diverts the SA bound ABD fusion protein from the lysosome compartment of the cell and back to the plasma membrane. At the plasma membrane, the SA dissociates from FcRn due to the neutral pH (e.g., pH 7.1-7.5) and the SA and ABD fusion protein are released back into the bloodstream. Therapeutics that include an ABD are capable of binding to albumin at a pH range of about pH 5.5 to about pH 7.2, and such therapeutics advantageously also undergo FcRn-driven endosomal albumin recycling and, thus, evade lysosomal degradation. Accordingly, therapeutics that include such ABDs advantageously exhibit longer serum half-lives than counterparts lacking ABDs. Such therapeutics are particular useful for the treatment of cancers, which are known to contain high levels of serum albumin and cancer cells metabolize albumin as an energy source.

As used herein, “serum albumin” or “SA” refers to a member of a family of globular proteins produced by the liver that functions primarily as a carrier protein of steroids, fatty acids, and thyroid hormones in the blood. Serum albumin also plays a major role in stabilizing extracellular fluid volume by contributing to oncotic pressure of plasma, and includes, but is not limited to, human serum albumin (HSA, GenBank Accession numbers: NM_000477 and NP_000468) and mouse serum albumin (MSA, Genbank accession numbers: NM_009654 and NP_0033784). The structure of albumin is characterized by several long a helices and contains eleven distinct binding domains for hydrophobic compounds. In humans, serum albumin is encoded by the ALB gene.

In some embodiments, the albumin binding domain binds albumin at a site that does not interfere with serum albumin binding to a neonatal Fc receptor (FcRn). By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two fusion proteins, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

In some embodiments, the albumin binding domain described or exemplified herein preferably specifically binds to serum albumin (e.g., HSA) at an epitope on the serum albumin molecule that does not participate in the interaction of the serum albumin molecule with the FcRn. Binding of the albumin binding domain to the serum albumin molecule, thus preferably does not substantially interfere with, inhibit, prevent, or otherwise reduce binding of the serum albumin molecule (e.g., HSA) with the FcRn. In some embodiments, the albumin binding domain does not compete with the FcRn for binding to the serum albumin molecule. In exemplary embodiments, the albumin binding domain does not sterically inhibit binding of serum albumin to the FcRn. Preferably, the albumin binding domain does not change the conformation of the serum albumin molecule such that the albumin cannot interact with the FcRn. In some embodiments, the albumin binding domain competes with FcRn binding to albumin.

In some embodiments, the albumin binding domain binds to serum albumin at a region that does not interfere with GP60 binding to serum albumin. Without being bound by any particular of operation, it is believed that allowing both the antibody-based drug conjugate and GP60 to bind to serum albumin enhances transport of the antibody-based drug conjugate to the tumor microenvironment.

In some embodiments, the albumin binding domain binds to serum albumin at a region that does not interfere with Secreted protein acidic and rich in cysteine (SPARC) binding to serum albumin. Without being bound by any particular of operation, it is believed that allowing both the antibody-based drug conjugate and SPARC to bind to serum albumin enhances retention time of the antibody-based drug conjugate in the tumor microenvironment and tumor uptake.

In some embodiments, the albumin binding domain binds SA (e.g., HSA) at a pH of 5.0+0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. In some embodiments, the albumin binding domain binds SA at a range of pH of about pH 5.5-about pH 7.2. In some embodiments, the SA binding moiety binds SA at a pH of 5.5.

In certain embodiments, the albumin binding domain is a human serum albumin (HSA) binding domain. HSA binding domains include, but are not limited to, albumin binding domains that can bind to an HSA molecule such as a whole HSA molecule or a fragment of an HSA. In some embodiments, the HSA binding domain also binds mouse serum albumin. In some embodiments, the HSA binding domain also binds cyno monkey albumin. In certain embodiments, the HSA binding domain does not bind to bovine serum albumin (BSA).

Albumin binding domains provided herein can include a variable heavy chain alone or a variable heavy chain in association with a variable light chain. In some embodiments, the albumin binding domain includes a variable heavy chain. In certain embodiments, the variable heavy chain includes a vhCDR1, vhCDR2, and vhCDR3 (variable heavy chain Complementary Determining Regions 1-3). In certain embodiments, the antigen binding domain also includes a variable light chain. In certain embodiments, the variable light chain includes a vlCDR1, vlCDR2 and vlCDR3 (variable light chain Complementary Determining Regions 1-3).

In some embodiments, the albumin binding domain includes the vhCDR1, vhCDR2, and vhCDR3 of an A10m3 variable heavy chain, as shown in Table 5. In certain embodiments, the albumin binding domain includes the vhCDR1, vhCDR2, and vhCDR3 of A10m3 as shown in Table 5.

In certain embodiments the albumin binding domain also includes a variable light chain. In some embodiments, the albumin binding domain includes the vlCDR1, vlCDR2, and vlCDR3 of an A10m3 variable light chain, as shown in Table 5. In certain embodiments, the albumin binding domain includes the vlCDR1, vlCDR2, and vlCDR3 of A10m3 as shown in Table 5.

TABLE 5

| | A10m3 Related Sequence | SEQ ID NO: |
|---|---|---|
| Variable | EVQLVESGGGLIQPGRSLRLSCAASGITFDDAVMHWVRQAPGKGLEWVAGISSNSG | 46 |
| heavy (vh) | YIGYADSVKGRFTISRDNAKNSLYLQMNRLRAEDTAVYYCVKGLYSNPRGGAFDIWG | |
| chain | QGTMVTVSS | |
| vhCDR1 | GITFDDAV | 47 |
| vhCDR2 | ISSNSGYI | 48 |
| vhCDR3 | VKGLYSNPRGGAFDI | 49 |
| Variable | SYVLTQPPSVSVAPGQTATITCGGNNIGTKSVHWYQQKPGQAPVLVVYADSDRPSGI | 50 |
| light (vl) | PERVSGSNSGNTATLTISRVEAGDEADYYCQVWDSRSDHLWVFGGGTKLTVLG | |
| chain | | |
| VlCDR1 | NIGTKS | 51 |
| VlCDR2 | ADS | 52 |
| vlCDR3 | QVWDSRSDHLWV | 53 |
| A10m3 scFv | EVQLVESGGGLIQPGRSLRLSCAASGITFDDAVMHWVRQAPGKGLEWVAGISSNSG | 54 |
| | YIGYADSVKGRFTISRDNAKNSLYLQMNRLRAEDTAVYYCVKGLYSNPRGGAFDIWG | |
| | QGTMVTVSSASTGGGGSGGGGSGGGGSVHSSYVLTQPPSVSVAPGQTATITCGGN | |
| | NIGTKSVHWYQQKPGQAPVLVVYADSDRPSGIPERVSGSNSGNTATLTISRVEAGDE | |
| | ADYYCQVWDSRSDHLWVFGGGTKLTVLG | |

In certain embodiments, the albumin binding domain (e.g., HSA binding domain) is an antibody or an antibody fragment. In some embodiments, the albumin binding domain (e.g., HSA binding domain) is an scFv.

In some embodiments where the ABD includes both a variable heavy chain and a variable light chain, the variable heavy chain and the variable light chain are attached to each other by a linker (e.g., an scFv linker). In certain embodiments, the linker is attached to the variable heavy chain at its C-terminus and the variable light chain at its N-terminus. Suitable linkers are described herein and Table 4. In some embodiments, the linker is a (Gly4Ser), linker, where x is 1, 2, 3, 4, 5, 6, 7, or 8. In certain embodiments, the linker is a (Gly4Ser) 5 linker.

In certain embodiments, the albumin binding domain also includes a variable heavy chain that includes the vhCDR1, vhCDR2 and vhCDR3 of A10m3 and a variable light chain that includes the vlCDR1, vlCDR2 and vlCDR3 of A10m3 (Table 5). In one embodiment, the albumin binding domain includes the variable heavy sequence and variable light sequence of the A10m3 ABD depicted in Table 5.

The albumin binding domain is attached to the antibody-based drug conjugate using any suitable method known in the art. In some embodiments, the albumin binding domain is attached to a targeting moiety of the antibody-based drug conjugate using a linker described herein. In exemplary embodiments, the linker is a peptide linker.

Additional useful albumin binding domains that are included in certain embodiments of the subject antibody-based drug conjugates described herein are disclosed in U.S. Pat. No. 11,028,166, which is incorporated by reference it its entirety, particular to disclosures relating to albumin binding domains.

### F. Methods of Making Anti-Based Drug Conjugates

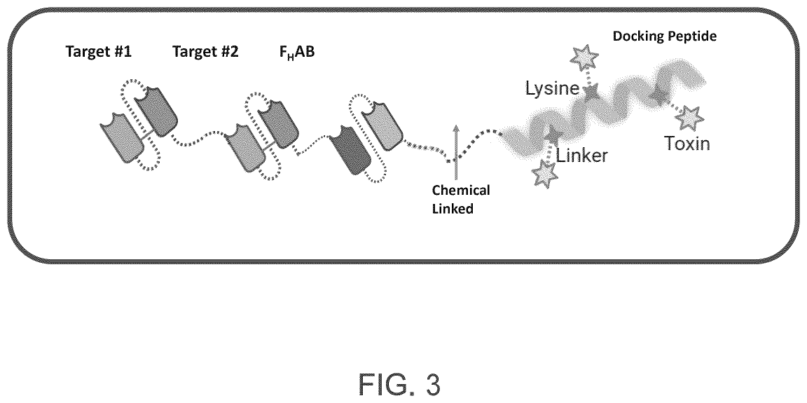

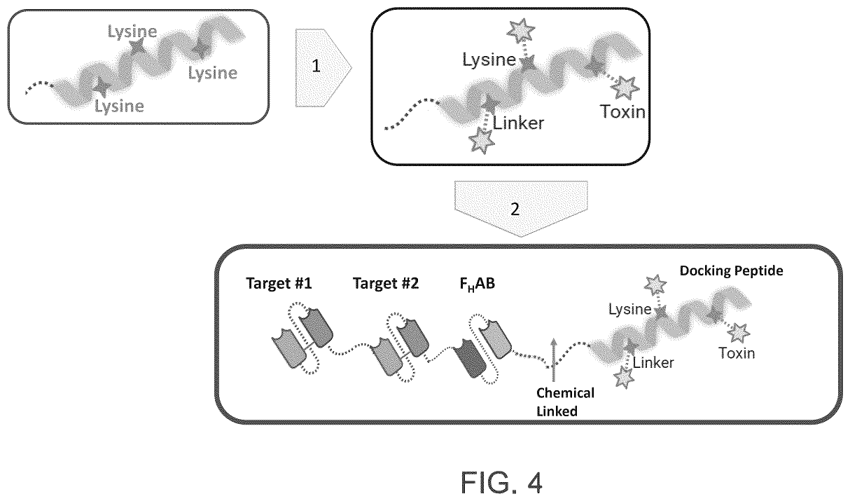

The anti-based drug conjugates provided herein can be made using any suitable technique. In some embodiments, the docking peptide with the drug moieties and the antibody-based targeting moiety are each separately made, and the docking peptide and antibody-based targeting moiety are subsequently linked to each other to form the subject antibody-based drug conjugate. See, e.g., Example 2 and FIGS. 3 and 4.

In some embodiments, the drug moieties are conjugated to the docking peptide via a cleavable linker using standard techniques. Standard techniques for conjugating drug moieties to the docking peptide and exemplary linkers are described herein.

The subject antibody-based targeting moiety and docking peptides provided herein can be made using any standard technique, including standard protein expression techniques. As is known in the art, nucleic acids encoding the antibody-based targeting moiety and docking peptides disclosed herein can be incorporated into expression vectors as is known in the art and depending on the host cells used to produce the antibody-based targeting moiety and docking peptides of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

The antibodies provided herein are made by culturing host cells comprising the expression vector(s) as is well known in the art.

### G. Methods of Determining Cytotoxic Effect of ScFv/Drug Conjugates

Methods of determining whether an antibody-based drug conjugate exerts a cytostatic and/or cytotoxic effect on a cell are known. Generally, the cytotoxic or cytostatic activity of an antibody-based drug conjugate can be measured by: exposing mammalian cells expressing a target protein of the antibody-based drug conjugate in a cell culture medium; culturing the cells for a period from between about 6 hours to about 5 days; and measuring cell viability. Cell-based in vitro assays can be used to measure viability (proliferation), cytotoxicity, and induction of apoptosis (caspase activation) of the antibody-based drug conjugate.

For determining whether an antibody-based drug conjugate exerts a cytostatic effect, a thymidine incorporation assay may be used. For example, cancer cells expressing a target antigen at a density of 5,000 cells/well of a 96-well plated can be cultured for a 72-hour period and exposed to 0.5 μCi of 3H-thymidine during the final 8 hours of the 72-hour period. The incorporation of 3H-thymidine into cells of the culture is measured in the presence and absence of the antibody-based drug conjugate.

For determining cytotoxicity, necrosis, or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that an antibody-based drug conjugate is useful in the treatment of cancers.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytotoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, J. Immunol. Methods 65:55-63 (1983)). In some embodiments, cell viability is measured by quantifying ATP using a luciferase reaction.

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Apoptosis can also be determined by measuring morphological changes in a cell. For example, as with necrosis, loss of plasma membrane integrity can be determined by measuring the uptake of certain dyes (e.g., a fluorescent dye such as, for example, acridine orange or ethidium bromide). A method for measuring apoptotic cell number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., Cancer Research 55:3110-16 (1995)).

In vivo, the effect of a therapeutic composition of the multispecific antibody of the invention can be evaluated in a suitable animal model. For example, xenogeneic cancer models can be used, wherein cancer explants or passaged xenograft tissues are introduced into immune compromised animals, such as nude or SCID mice (Klein et al., Nature Medicine 3:402-408 (1997)). Efficacy can be measured using assays that measure inhibition of tumor formation, tumor regression or metastasis, and the like.

The therapeutic compositions used in the practice of the foregoing methods can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate or TRIS-buffered saline solutions, bacteriostatic water, and the like (see, generally, Remington's Pharmaceutical Sciences 16th Edition, A. Osal., Ed., 1980).

### H. Uses and Treatment Modalities

The subject docking peptides, antibody-based drug conjugates and compositions provided are useful in any suitable method of treatment where targeted, efficient, controlled delivery of drug moieties is needed.

In some embodiments, the subject antibody-based drug conjugates and compositions provided are useful in methods for treating a cancer. The subject methods can be used to treat any suitable cancer including, but not limited to brain cancer, head and neck cancer, lung cancer, pleura cancer, breast cancer, gastric cancer, liver cancer, colon cancer, pancreatic cancer, renal cancer, ovarian cancer, prostate cancer, skin cancer, thyroid cancer, bone cancer, soft tissue cancer, and neural cancer.

In certain embodiments, the subject antibody-based drug conjugates and compositions provided here are used to treat and/or prevent an infectious disease. In exemplary embodiments, the infectious disease is a local or systemic viral infection such as encephalitis, influenza, common cold, immunodeficiency, and herpes viral infections. In certain embodiments, the infectious disease is caused by a bacterium, virus, protozoan, helminth, or other microbial pathogen. In particular embodiments, the infection to be treated or prevented is caused by a hepatitis virus, a human immunodeficiency virus (HIV), a human T-lymphotrophic virus (HTLV), a herpes virus, an Epstein-Barr virus, or a human papilloma virus.

In the methods provided herein, therapy is used to provide a positive therapeutic response with respect to a disease or condition. By “positive therapeutic response”, the therapy is intended to provide an improvement in the disease or condition, and/or an improvement in the symptoms associated with the disease or condition. For example, a positive therapeutic response would refer to one or more of the following improvements in the disease: (1) a reduction in the number of tumor cells; (2) an increase in tumor cell death; (3) inhibition of tumor cell survival; (4) inhibition (i.e., slowing to some extent, preferably halting) of tumor growth; (5) an increased patient survival rate; and (6) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, positron emission tomography (PET) scan imaging, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

An improvement in the disease may be characterized as a complete response. By “complete response” is intended an absence of clinically detectable disease with normalization of any previously abnormal radiographic studies, bone marrow, and cerebrospinal fluid (CSF) or abnormal monoclonal protein in the case of myeloma.

Such a response may persist for at least 4 to 8 weeks, or sometimes 6 to 8 weeks, following treatment according to the subject methods. Alternatively, an improvement in the disease may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, the specific cancer type in question, and the particular composition or route of administration selected.

### I. Pharmaceutical Formulations, Administration and Dosing

The antibody-based drug conjugates and compositions provided herein may be formulated for administration to a subject using techniques known to those skilled in the art. Formulations comprising such antibody-based drug conjugates and compositions include a pharmaceutically acceptable carrier. Exemplary carriers include, but are not limited to, any of several standard pharmaceutical carriers such as sterile phosphate or TRIS-buffered saline solutions, bacteriostatic water, and the like (see, generally, Remington's Pharmaceutical Sciences 16th Edition, A. Osal., Ed., 1980).

The antibody-based drug conjugates and compositions provided herein may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing one or more antibody-based drug conjugate), it will generally be formulated in a unit dosage injectable form (solution, suspension, emulsion).

The antibody-based drug conjugates and compositions provided herein can be formulated for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

In some embodiments, the antibody-based drug conjugates and compositions provided herein are provided as sterile liquid preparations, e.g., isotonic aqueous solutions, suspensions, emulsions, dispersions, or viscous compositions, which may in some aspects be buffered to a selected pH. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

In certain embodiments, the antibody-based drug conjugates and compositions provided herein are provided as an injectable solution. Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

### J. Administrative Modalities

The subject antibody-based drug conjugate and compositions are administered to a subject, in accord with known methods, such as intravenous administration as a bolus or by continuous infusion over a period of time, by intramuscular, intraperitoneal, intracerobrospinal, subcutaneous, intra-articular, intrasynovial, intraspinal, intrathecal, oral, topical, or inhalation routes. In an exemplary embodiment, the antibody-based drug conjugates and compositions are administered by intravenous infusions or intratumoral/tissue injections.

In some embodiments, the size or timing of the doses is determined as a function of the particular disease or condition in the subject. It is within the level of a skilled artisan to empirically determine the size or timing of the doses for a particular disease in view of the provided description.

### EXAMPLES

### Example 1: Making Docking Peptides

### Computational Methods

All molecular modeling operations were performed with the Maestro (Schrödinger) molecular modeling package and AMBER18 running on an Exxact Workstation with 48 CPU and a GPU running centOS 8. Amino acid mutations, side chain rotamer search and energy optimizations were performed using a Prime program that uses a Maestro interface. Helix properties were calculated using Multiple Sequence viewer in Maestro and online servers such as Peptide Calculator, Peptide Cutter, IEDB and Prosper servers. An Immunogenicity prediction algorithm developed at Abzena was also applied for consensus prediction. Molecular Dynamics (MD) simulation calculations were performed on one 21 amino acid long modeled helix system with 3 Lys residue arrangement to confirm the stability of the helix in solution over a microsecond time scale.

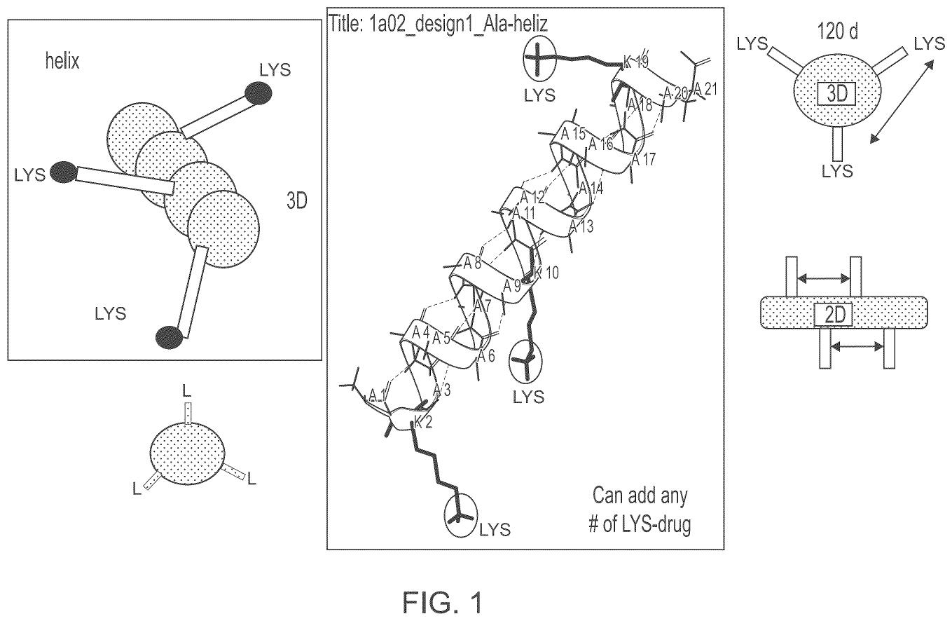

Protein structures with longer and higher helical content and resolution below 2 Å were downloaded from the Protein Data Bank (PDB). A set of preferred amino acids with higher helix propensity, preferred acceptance at N- or C-terminal end and excluding those with positively charged side chains, except for Lys, were selected for improved aqueous water solubility. Hundreds of helical peptide sequences of different lengths were designed using a helix from PDB code 1A02 as a template. While designing spacing of Lys residues over the length of peptide, a careful consideration was given to introduce >10 Å separation between Lys side chain amines in 3D, while on a 2D sequence level, two nearest Lys would be separated by at least 2 non-Lys amino acids (see FIG. 1), which led to helices of 21 amino acids in length for 3-Lys designs (see FIG. 2). Selected sequences with desired 2D and 3D design features were used for various property predictions such as CD4 T cell immunogenicity (IEDB and i-Tope servers), proteolytic cleavage by trypsin and chymotrypsin (Expasy webserver) and metalloproteinase-2 and -9 (Prosper webserver), hydrophilicity/hydrophobicity and acidic/basic/neutral content.

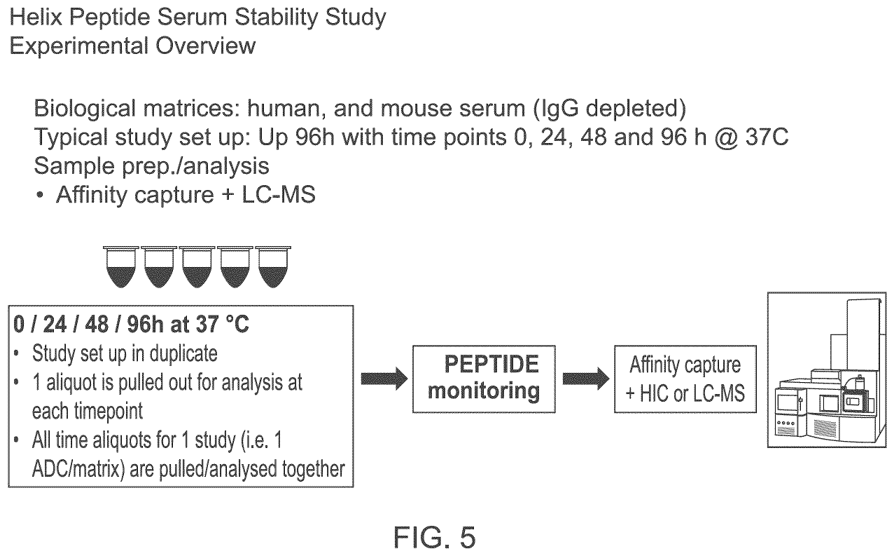

Candidate docking peptides were assessed for serum stability using the experimental design shown in FIG. 5. As shown in FIG. 6A, exemplary subject docking peptides, including DNIDKEHNDEKAADSDKEHDE (SEQ ID NO:5) were stable in human serum even after 5 days. Docking peptide DNIDKEHNDEKAADSDKEHDE E (SEQ ID NO:5) was further assessed for conjugation to MMEA. As shown in FIG. 6B, the DNIDKEHNDEKAADSDKEHDE (SEQ ID NO:5) docking peptide attached an antibody based targeting moiety (referred to as “scFv5”) comprising two anti-HER2 scFvs (4D5 mouse sequence, J Immunol. 2009 Nov. 1; 183 (9):5563-5574) and an anti-albumin scFv exhibited highly efficient conjugation to MMEA, wherein the 3 lysines in the alpha helical peptide are conjugated with 3 MMEA provided herein.

### Example 2: Methods of Making Antibody-Based Drug Conjugates

FIG. 4 provides an exemplary schematic for making the subject antibody-based drug conjugates. docking peptides bearing a terminal azido-lysine residue for conjugation to antibody-based targeting moieties, and a number of natural lysines for conjugation to drug moieties, were made as discussed in Example 1 (step 1) and conjugated to cytotoxic payloads to form a drug conjugate moiety (step 2). Drug moieties (e.g., MMAE tubulin inhibitor) were conjugated to the docking peptides using the cleavable linker NHS-PEG4-val-cit-PAB using standard techniques. NHS-PEG4-val-cit-PAB is an N-hydroxysuccinimide group linked to four repeating units of polyethylene glycol (PEG) and a cathepsin B cleavable dipeptide linked to p-aminobenzyl (PAB). The antibody targeting moiety was functionalized on a terminal cysteine position with a DBCO-PEG4-maleimide. The DBCO-PEG4-maleimide is a dibenzocyclooctyne group linked to four repeating units of polyethylene glycol (PEG), and a maleimide group. The drug conjugate moieties were then linked to a DBCO functionalized antibody-based targeting moiety via strain promoted alkyne-azide cycloaddition reaction (SPAAC) (step 3). For the antibody-based targeting moieties, scFvs (e.g., targeting moiety and optional albumin binding domain) were linked to each other using a peptide linker (e.g., a glycine serine linker). See, e.g., Ou et al., PLOS ONE 13 (10): e0206246.

### Example 3: Functional Assessment of Antibody-Based Drug Conjugates

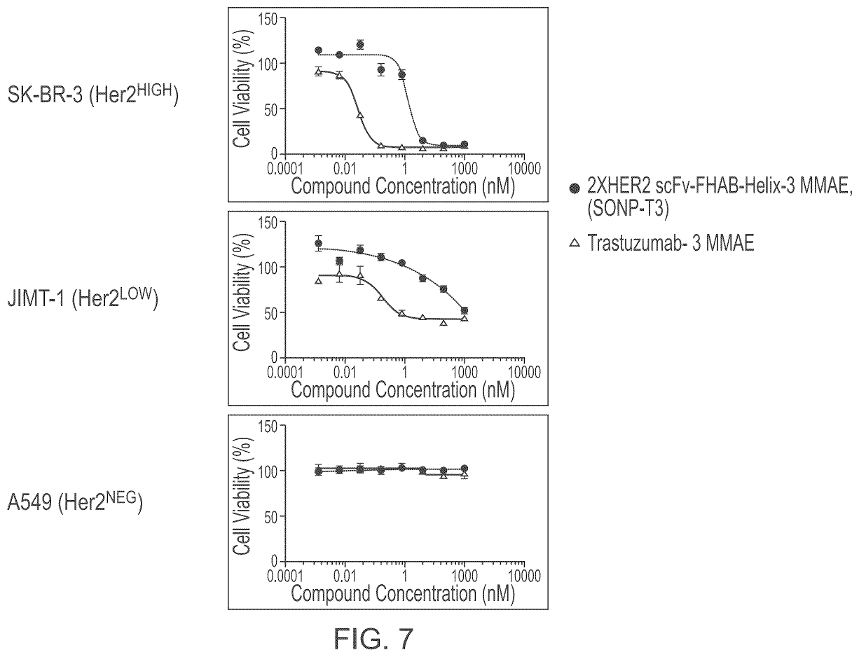

The potency of an exemplary subject antibody-based conjugate (referred to as “scFv5-peptide-3×MMAE” or “SONP-T3”) was functionally assessed in a cell viability and proliferation assay using various HER2-expressing cell lines including SKBR3 (HER2^{High}), JIMT-1 (HER2^{Low}), and A549 (HER2^{Neg}) control. The exemplary scFv5-peptide-3×MMAE included 1) an “scFv5” antibody-based targeting moiety comprising two anti-HER2 scFvs (4D5 mouse sequence, J Immunol. 2009 Nov. 1; 183(9):5563-5574) and an anti-albumin scFv; and 2) a subject docking peptide (SEQ ID NO:5) with three lysines wherein an MMAE payload is attached to each of the lysines. As shown in FIG. 7, the scFv5-peptide-3×MMAE exhibited higher killing on cells expressing higher levels of HER2 compared to cells expressing lower levels of HER2, and exhibited no off-site toxicity on HER2^{Neg} cells.

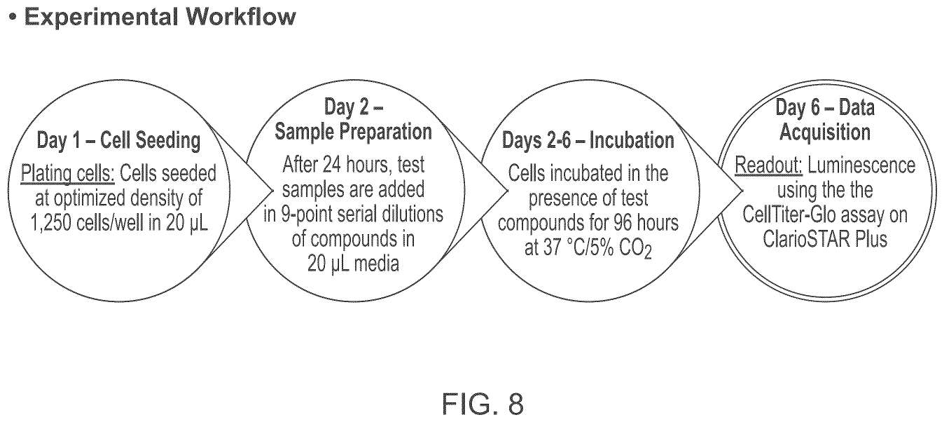

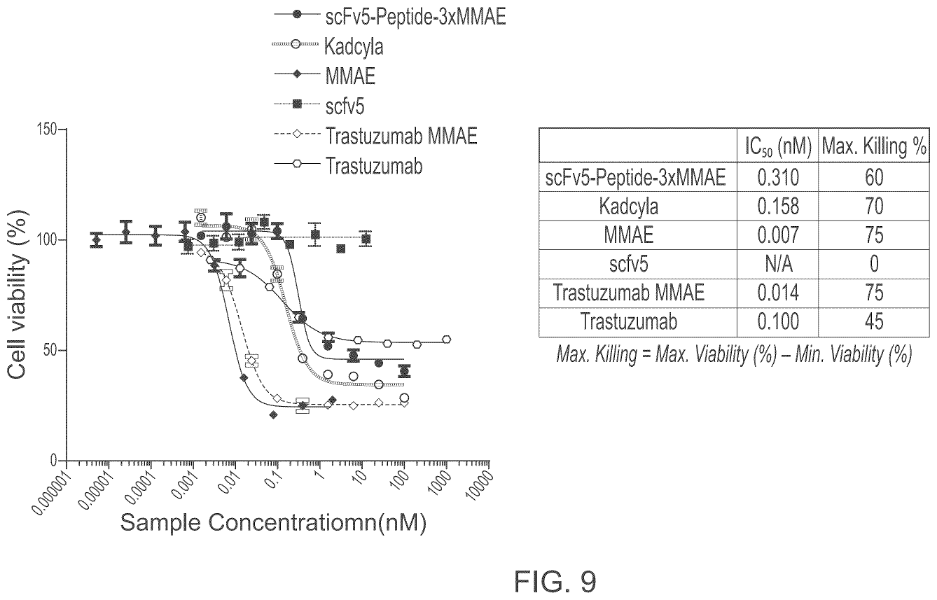

The potency of scFv5-peptide-3×MMAE (also referred to as “SONP-T3”) was further tested using HER2 positive BT-474 (HER2^{High}) carcinoma cells according to the experimental design shown in FIG. 8. As shown in FIG. 9, scFv5-peptide-3×MMAE exhibited significant killing of BT-474 cells.

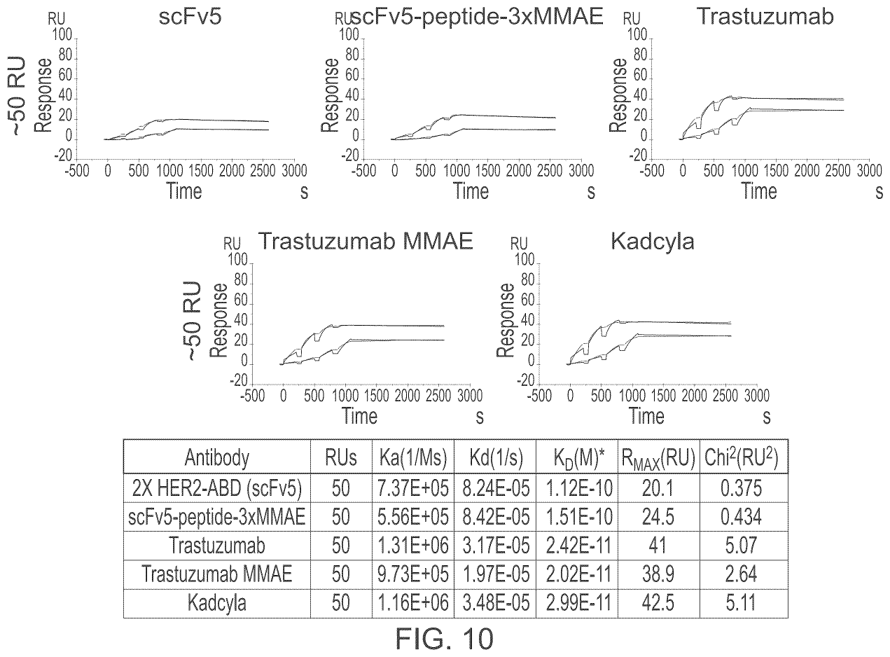

The binding of HER2 binding proteins, including scFv5, was assessed. Conjugation to ADC did not appear to affect the binding of scFv5 and Trastuzumab® to HER2 ligand (FIG. 10).

### Example 4: In Vivo Assessment of Antibody-Based Drug Conjugates

In vivo antitumor activity and toxicity of exemplary SONP-T3 (also referred to as “scFv5-peptide-3×MMAE” herein) was further evaluated in a BT-474 subcutaneous breast cancer mouse model. Included in this study was Trastuzumab (IgG)-3×MMAE, which included the same attachment to LYS, linker chemistry, cleavable sequence and toxin (MMAE) as SONP-T3, Kadcyla having the same anti-HER2 IgG conjugated to 3×DM1, and a scFv5-docking peptide (SEQ ID NO:5) without toxin (referred to as “SONP-NT”). A summary of the experimental design of this study is shown in Table 6 below.

TABLE 6

| | | | | | Toxicity |
|---|---|---|---|---|---|
| | | | | Tumor | Testing |
| | Dose | | Dosing ** | Volume | 1 day after 4^{th} |
| | (mg/kg) | Number of | Intervals | And | dose & |
| | Final | mice per | @ TV | Body | 10 days after |
| Test substance | Toxin* | group | 200 mm3 | Weight | 4^{th} dose |
| Placebo | 0.2 ml | 8 | Q4Dx4 IV | 2X week | Blood |
| buffer | | | | | Chemistry |
| | | | | | Hematological |
| | | | | | Profile |
| scFv5-pepetide | 0.2 ml | 8 | Q4Dx4 IV | 2X week | Blood |
| (no toxin) or | | | | | Chemistry |
| “SONP-NT” | | | | | Hematological |
| | | | | | Profile |
| scFv5-peptide- | 5, 10, 15* | 8 | Q4Dx4 IV | 2X week | Blood |
| 3xMMAE (DAR3) | | | | | Chemistry |
| or “SONP-T3” | | | | | Hematological |
| | | | | | Profile |
| Trastuzumab- | 5, 10* | 8 | Q4Dx4 IV | 2X week | Blood |
| PEG4-MMAE | | | | | Chemistry |
| (DAR3) | | | | | Hematological |
| | | | | | Profile |
| Kadcyla | 5, 10* | 8 | Q4Dx4 IV | 2X week | Blood |
| DM1 (DAR3) | | | | | Chemistry |
| | | | | | Hematological |
| | | | | | Profile |

*Adjusted based on MW to have the same toxin number/construct. SONNET ADC 88 kD vs ADC IgG 150 kD.
** Q4Dx4: total of 4 administration (one injection per day every four days).

### Experimental Design and Treatments

a. Randomization & Treatment

Animals were randomized based on their individual tumor volume. Randomization was performed when values reached a mean of 150-250 mm3. The treatment schedule was Group 1-9 animals received four IV administrations of vehicle, i.e., one injection per day every four days. Tumor volume and body weight were taken twice a week.

b. Sample Preparation & Blood Collection

Eight mice for each construct tested were divided into subgroups A (4 mice) and B (4 mice). For the subgroups A, 60 μl of whole blood was collected by jugular vein puncture at 4 time points Day randomization (Dr) after first dose (baseline, Dr+8, Dr+13 and Dr+23). Blood was collected into collection tubes with anticoagulant (K2 EDTA) for the hematological profile type counting.

For the subgroups B, 120 μl of whole blood was collected by jugular vein puncture at 2 timepoints (Dr+13 and Dr+23). For the baseline, 4 mice of residual mice (non-randomized mice) was collected at Dr them 4 mice of subgroup B of group 1 (vehicle) was collected at Dr+13 and Dr+23. Blood was collected into collection tubes with anticoagulant (lithium heparin) for biochemical analysis parameters (ALAT, ASAT, Creatine, Creatine kinase, Total bilirubin).

c. Animal Monitoring

Body weights were measured at least twice a week, and the length and width of the tumor were measured at least twice a week with calipers. Hematological profiles and changes in blood chemistry were monitored at day 0 (no treatment) baseline/control compared to day 13, one day after the 4th dose, and day 23, ten days after the 4th last dose.

d. Humane Endpoints

Humane endpoints requiring euthanasia have been established as tumors exceeding 10% of normal body weight or 1000 mm3 (tumors in mice).

### Results

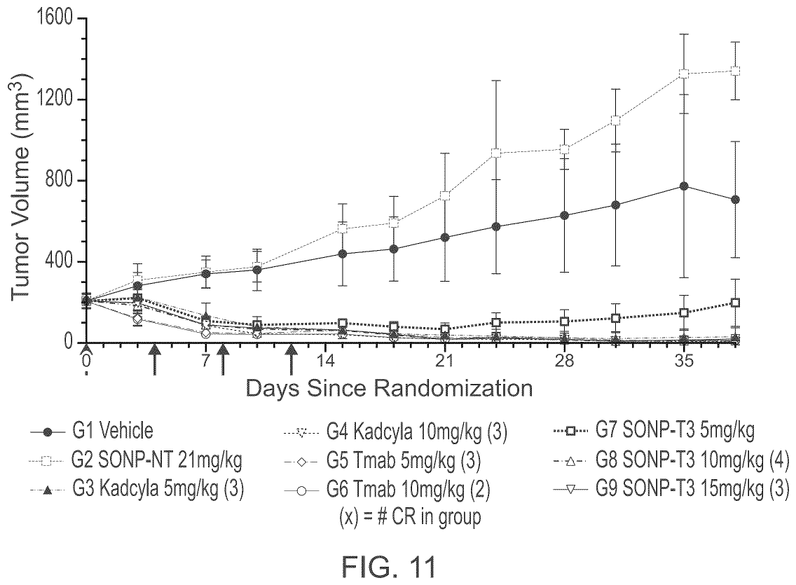

As shown in FIG. 11, SONP-T3 shows similar efficacy to Kadcyla and Trastuzumab in preventing tumor growth.

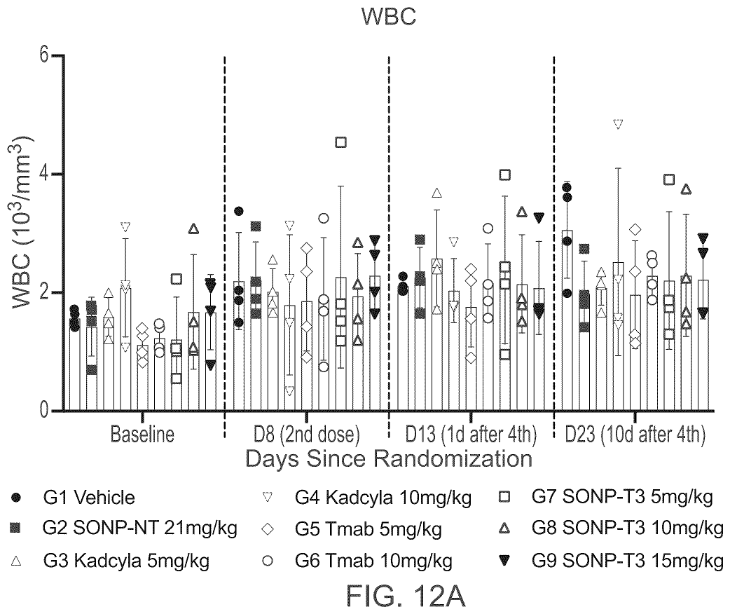

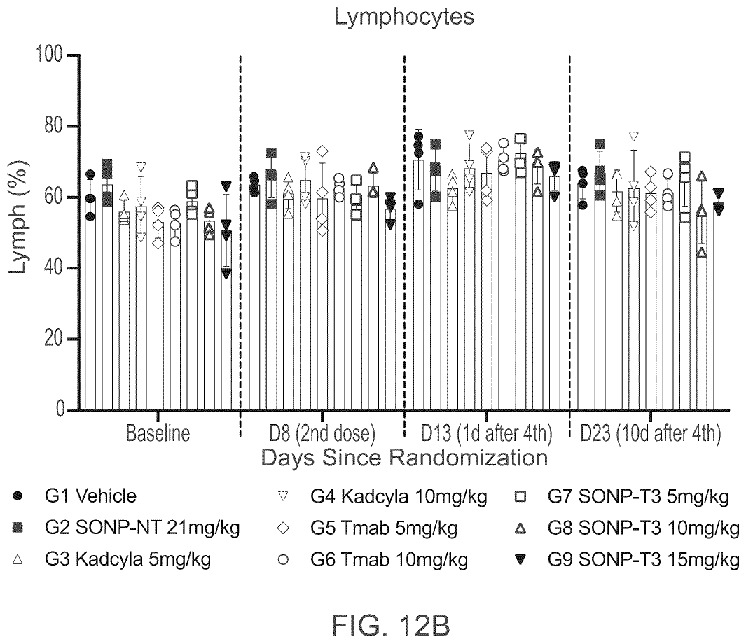

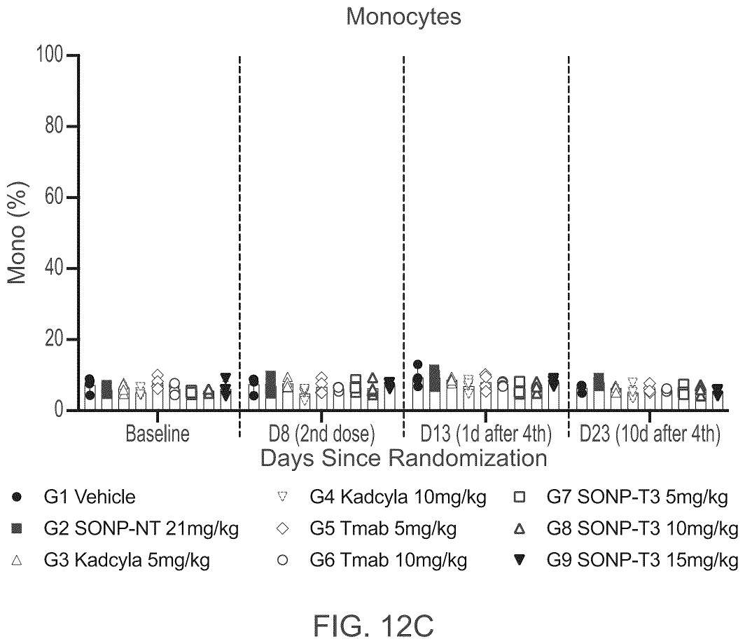

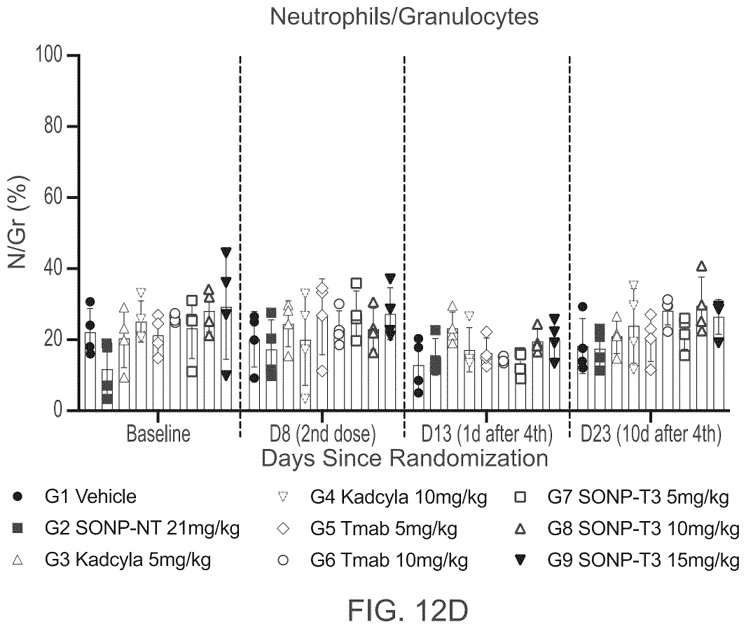

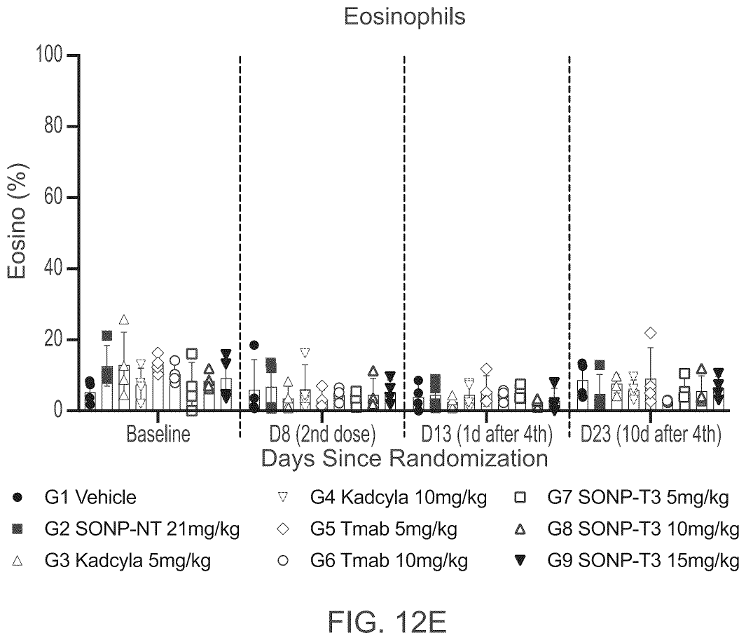

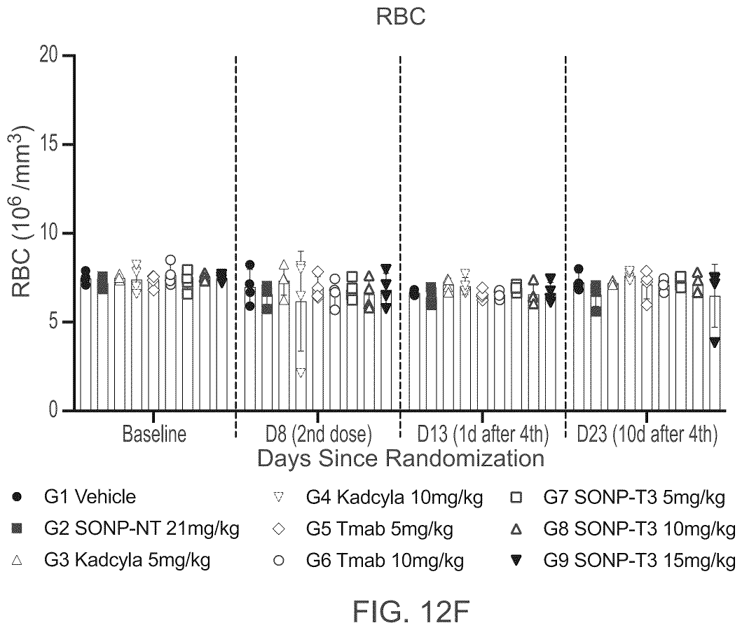

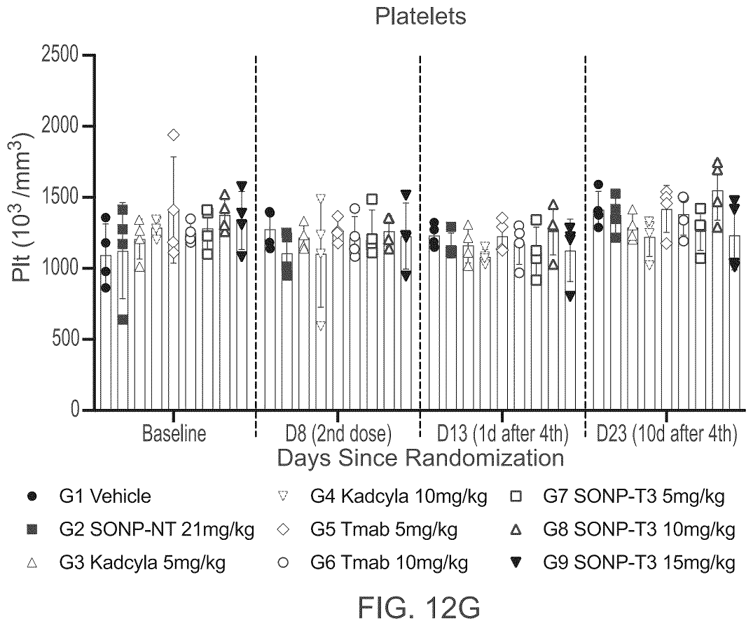

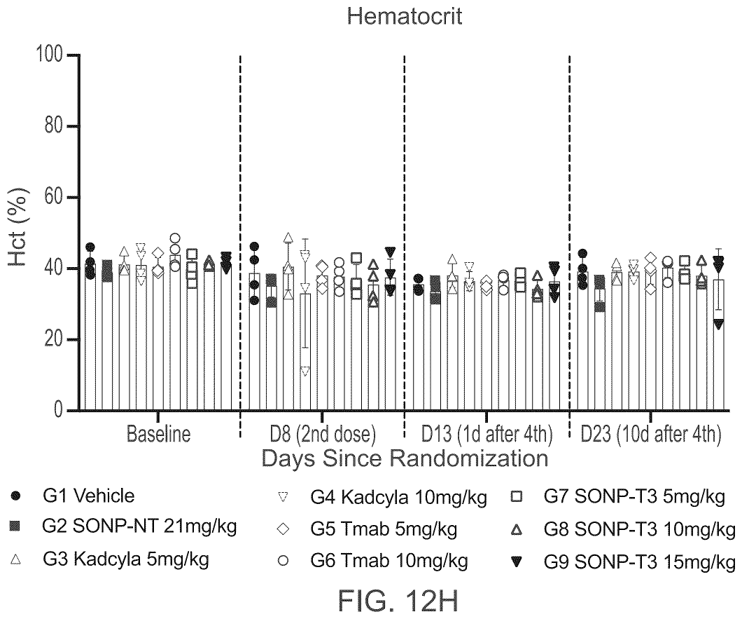

ADCs can cause many types of hematological toxicities, including: anemia, neutropenia, pancytopenia, thrombocytopenia, leukopenia, and lymphopenia. Thus, hematological profiles were taken during this study to assess the effects of the various constructs on the following: white blood count (WBC), lymphocytes, monocytes, neutrophils/granulocytes, eosinophils, red blood cells, platelets, hematocrit, and hemoglobin. As shown in FIGS. 12A-121, SONP-T3 did not show any hematological toxicities as compared to baseline values.

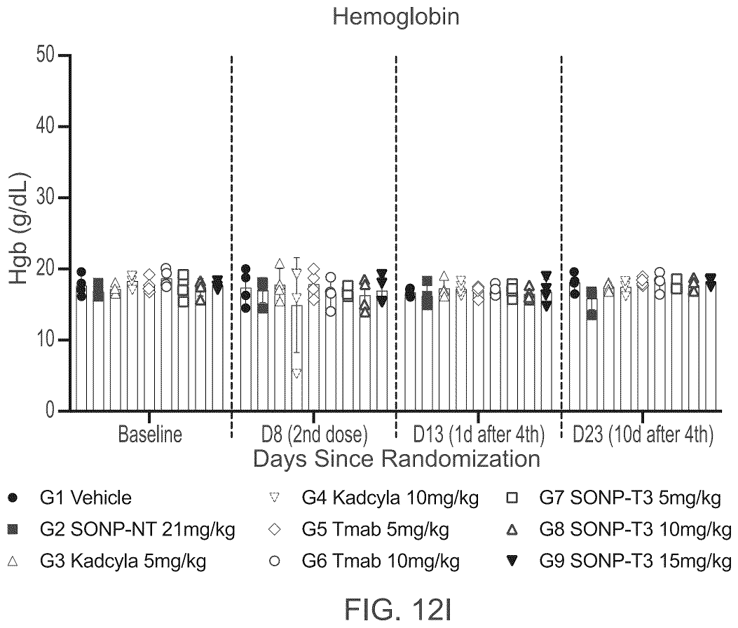

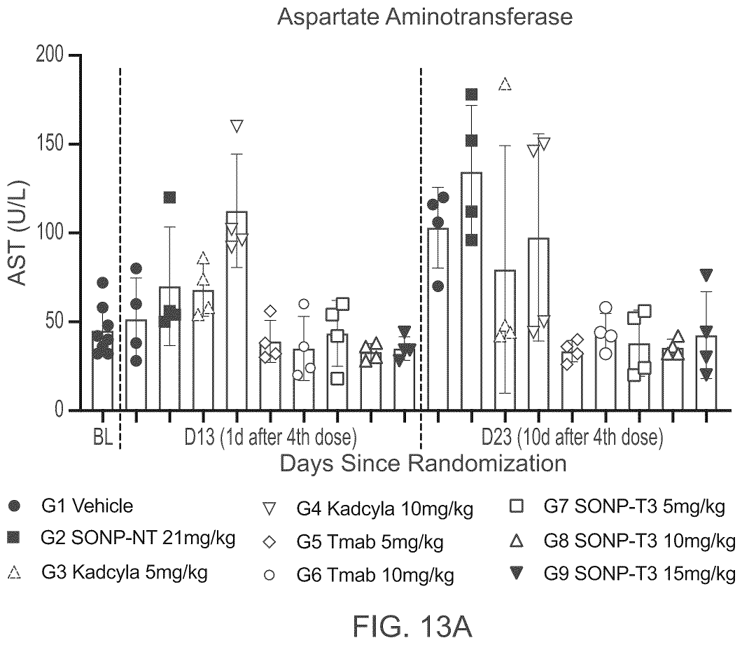

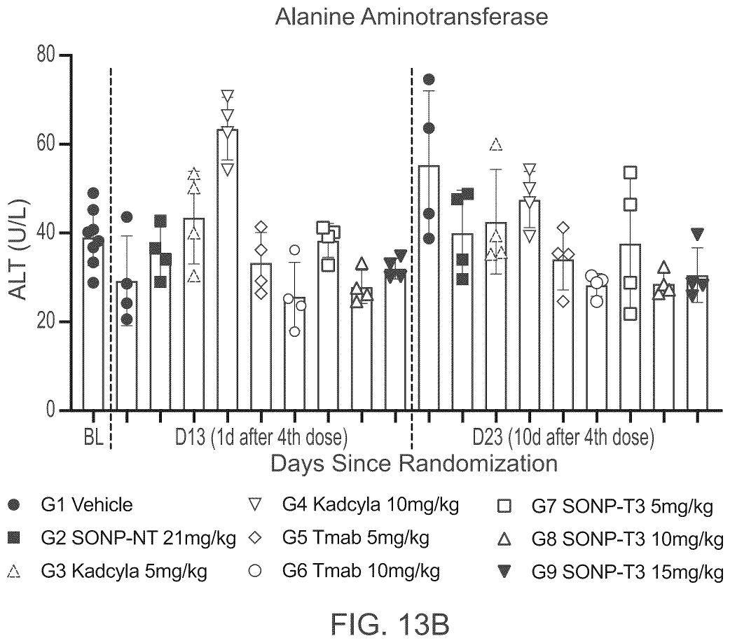

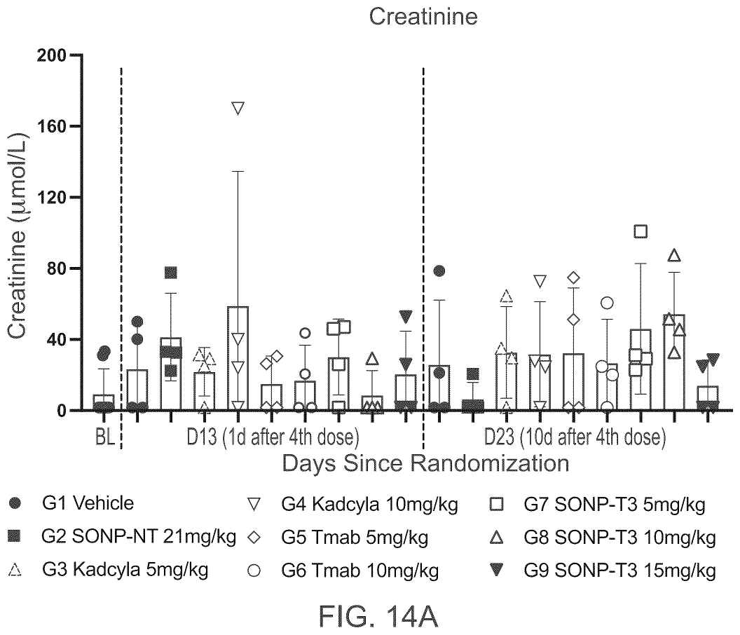

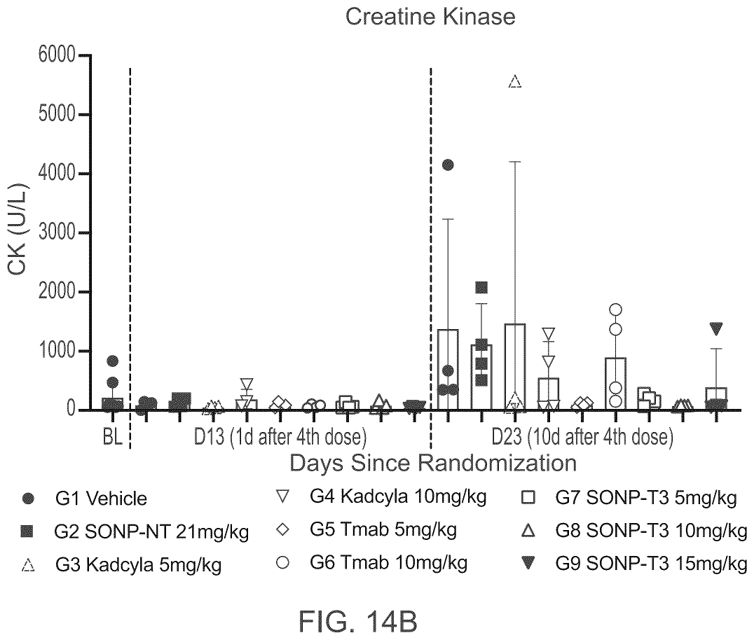

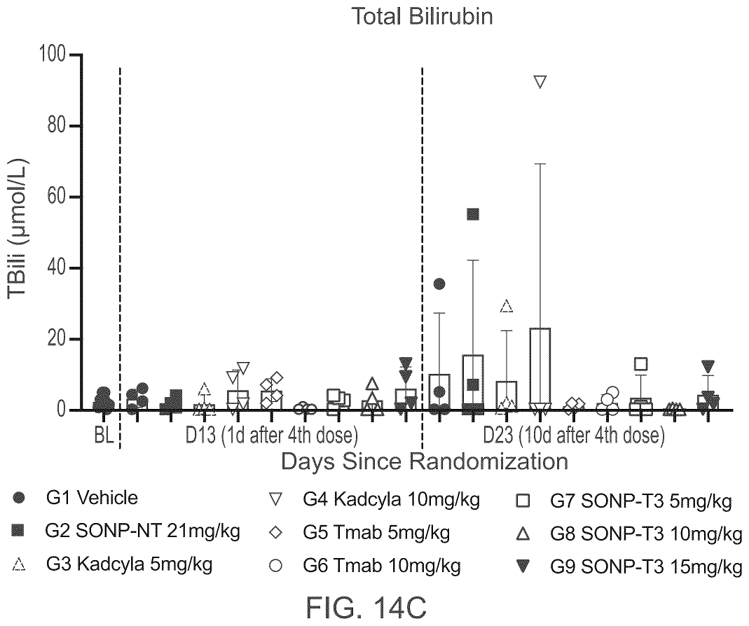

ADCs can potentially have adverse events that require therapeutic dose reduction or treatment discontinuation. Their toxicity is primarily due to the premature release of cytotoxic payloads into the patient's blood, which can be caused by unstable linkers connecting the antibody and the cytotoxic payload. Thus, animals in this study were also assessed during this study for elevated liver enzymes aspartate aminotransferase (AST) and alanine aminotransferase (ALT) (FIGS. 13A and 13B), which may indicate hepatotoxicity; creatine toxicity for nephrotoxicity (FIG. 14A); creatine kinase (CK) levels for muscle and/or kidney damage (FIG. 14B), and total bilirubin levels that may reflect central nervous system damage (FIG. 14C). As shown in FIGS. 13 and 14, SONP-T3 showed values similar to baseline control.

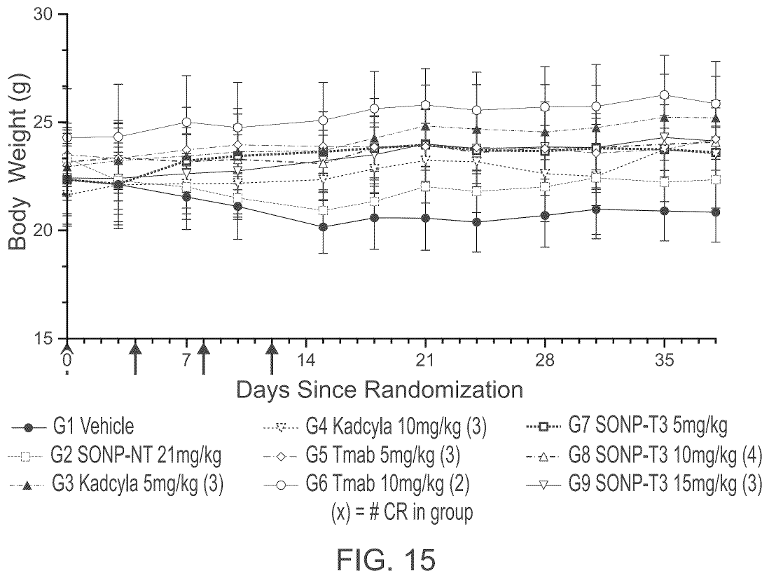

Body weights were monitored twice a week during the course of this study (FIG. 15). No change/loss (>10%) was observed throughout the study, indicating no overall toxicity for SONP-T3.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

1.-48. (canceled)

49. A composition comprising:
a) a targeting moiety; and
b) a drug conjugate moiety comprising:
i) a docking peptide comprising the amino acid sequence of SEQ ID NO:5; and
ii) one or more drug moieties,
wherein each of the one or more drug moieties is attached to a lysine residue of the docking peptide, and
wherein the targeting moiety is attached to the drug conjugate moiety by a linker.

50. The composition of claim 49, wherein the targeting moiety is a tumor targeting moiety.

51. The composition of claim 49, wherein the tumor targeting moiety binds to one or more of the following tumor antigens: CD33, CD30, HER2, HER3, CD22, CD79b, NaPi2b, glycoprotein NMB, CD19, CD138, PSMA, CEA, guanlyl cyclase C, CD198, EGFR, CD52, CD74, FOLR1, CD37, mesothelin, CECAMS, LAMP1, GPNMB, CD56, TROP2, Mucin 1, STEAP1, Mesotehlin, Nectin 4, ENP3, guanylyl cyclase C (GCC), SLC44A4, NaPi2b, CD70 (TNFSF7), Cap, 5T4, SLTRK6, SC-16, LIV-1 (ZIP6), and P-Cadherin.

52. The composition of claim 50, wherein the tumor targeting moiety comprises one or more anti-tumor antigen scFvs.

53. The composition of claim 52, wherein the anti-tumor antigen scFvs comprises two anti-tumor antigen scFvs that each bind to the same tumor antigen.

54. The composition of claim 52, wherein the anti-tumor antigen scFvs comprises two anti-tumor antigen scFvs that each bind to a different tumor antigen.

55. The composition of 49, wherein the composition further comprises an anti-human serum albumin scFv, wherein the anti-human serum albumin scFv is attached to the targeting moiety and the drug conjugate moieties by linkers.

56. The composition of claim 49, wherein each of the one or more drug moieties comprises a nuclear localization signal.

57. The composition of 49, wherein each of the one or more drug moieties comprises a nuclear localization signal.

58. The composition of claim 49, wherein the one or more drug moieties are each attached to a lysine residue of the docking peptide by a linker.

59. The composition of claim 58, wherein the linker is a cleavable linker.

60. The composition of claim 59, wherein the cleavable linker is a cathepsin B cleavable linker.

61. The composition of claim 58, wherein the linker is selected from a hydrazone linker, a peptide linker, a disulfide linker and a thioether linker.

62. The composition of claim 49, wherein the drug moiety is selected from the group consisting of a microtubule inhibitor, a DNA cleavage enzyme, an Akt inhibitor, a DNA intercalator, a DNA transcription factor inhibitor, a DNA cross linker, and a Dihydrofolate Reductase (DHFR) inhibitor.

63. The composition of claim 55, wherein anti-human serum albumin scFv has the amino acid sequence of SEQ ID NO: 54.

64. A method of treating cancer in a patient in need thereof comprising administering to the patient the composition of claim 49.

65. A docking peptide comprising the amino acid sequence of SEQ ID NO:5.

66. The docking peptide of claim 65, wherein the docking peptide further comprises a lysosome targeting peptide.

67. The docking peptide of claim 65, wherein the docking peptide further comprises one or more drug moieties, wherein each of the drug moieties is attached to one of the lysine residues of the docking peptide.

68. The docking peptide of claim 67, wherein the one or more drug moieties each further comprises a nuclear localization signal.

69. A nucleic acid encoding the docking peptide of claim 65.

70. An expression vector comprising the nucleic acid of claim 69.

71. A host cell comprising the expression vector of claim 70

72. A method of making a docking peptide comprising culture the host cell of claim 71 under conditions for expressing the docking peptide and recovering the docking peptide.
